(19) United States
(12) Patent Application Publication
(10) Pub. No.: US 2024/0226491 A9
(43) Pub. Date: Jul. 11, 2024

(54) SYSTEMS, METHODS, AND APPARATUS FOR PRODUCING NITRIC OXIDE

(71) Applicant: VERO BIOTECH INC., Atlanta, GA (US)

(72) Inventors: Jason WHITE, Marietta, GA (US); Robyn Michelle MCCLURE, Atlanta, GA (US); Michael Albert AXELROD, Roswell, GA (US); Thorsten SCHMIDT, Decatur, GA (US); Jin Eul SONG, Chamblee, GA (US); Mary Virginia SMITH, Lilburn, GA (US)

(21) Appl. No.: 18/491,482

(22) Filed: Oct. 20, 2023

Related U.S. Application Data
(60) Provisional application No. 63/418,426, filed on Oct. 21, 2022.
(60) Provisional application No. 63/418,430, filed on Oct. 21, 2022.

(51) Int. Cl. A61M 16/00; A61M 16/06; A61M 16/10

(52) U.S. Cl. CPC A61M 16/022; A61M 16/06; A61M 16/1075; A61M 2202/0275; A61M 2205/3379

(57) ABSTRACT

Disclosed herein is a console comprising: at least one air inlet; a first receptacle configured to host a first source of nitric oxide; a second receptacle configured to host a second source of nitric oxide; a controller configured to selectively couple the at least one air inlet to one of the first receptacle or the second receptacle to deliver the nitric oxide from the first or the second source; and an outlet coupled to the first and/or second receptacle and configured to deliver nitric oxide to a subject.

### CROSS-REFERENCE TO RELATED APPLICATIONS

This application claims the benefit of U.S. Provisional Application No. 63/418,426, filed Oct. 21, 2022, and of U.S. Provisional Application No. 63/418,430, filed Oct. 21, 2022, the contents of which are incorporated herein by reference in their entirety.

### FIELD

Some aspects described herein relate to a medical device and, more particularly, to systems and methods for producing and delivering a gas that includes nitric oxide.

### BACKGROUND

Some aspects described herein relate to the production of nitric oxide (NO), which is then typically delivered to a patient in a medical setting.

Nitric oxide is a vasodilator indicated to improve oxygenation and reduce the need for extracorporeal membrane oxygenation, particularly in term and near-term neonates with hypoxic respiratory failure associated with clinical or echocardiographic evidence of pulmonary hypertension in conjunction with ventilatory support. Low concentrations of inhaled nitric oxide can also prevent, reverse, or limit the progression of disorders, which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, hyaline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism and idiopathic or primary pulmonary hypertension or chronic hypoxia.

Generally, nitric oxide can be inhaled or otherwise delivered to the individual's lungs. Providing a therapeutic dose of nitric oxide could treat a patient suffering from a disorder or physiological condition that can be mediated by inhalation of nitric oxide or supplement or minimize the need for traditional treatments in such disorders or physiological conditions. A significant challenge for nitric oxide delivery is that the presence of even trace amounts of oxygen (O₂) in nitric oxide-containing gasses can oxidize the nitric oxide, producing nitrogen dioxide (NO₂). Unlike nitric oxide, nitrogen dioxide, which forms nitric acid and nitrous acid in the lungs, is highly toxic at levels as low as a few parts per million. Accordingly, some aspects described herein relate to systems and methods to produce nitric oxide on-demand, which reduces the duration over which nitric oxide is exposed to oxygen.

To ensure continuous delivery, some known nitrogen oxide delivery devices require the user to follow a sequence of steps to hand off the delivery of nitric oxide from a first delivery system to a second delivery system when the first delivery system nears depletion. Some aspects described herein describe systems and methods that improve the continuity of nitric oxide delivery.

Some known nitric oxide delivery devices are sensitive to orientation and can malfunction if moved or tilted. Accordingly, a need exists for systems and apparatus that are resistant to orientation-related malfunctions. This need and all other needs are at least partially addressed by this disclosure.

### SUMMARY

The present disclosure is directed to a console comprising: at least one air inlet; a first receptacle configured to host a first source of nitric oxide; a second receptacle configured to host a second source of nitric oxide; a controller configured to selectively couple the at least one air inlet to one of the first receptacle or the second receptacle to deliver the nitric oxide from the first or the second source; and an outlet coupled to the first and/or second receptacle and configured to deliver nitric oxide to a subject.

Also disclosed herein are methods comprising: routing air from at least one air inlet through a first cassette that produces nitric oxide gas; detecting a fill state of the first cassette; and wherein the fill state of the first cassette is close to or below a threshold value, automatically re-routing air from the at least one air inlet through a second cassette that produces nitric oxide.

Still further disclosed herein is an apparatus comprising: a frangible vessel containing dinitrogen tetroxide; a reservoir, the frangible vessel disposed within the reservoir, the reservoir configured to contain the dinitrogen tetroxide when the frangible vessel is broken; and an outlet, the outlet disposed above a wall of the reservoir, the frangible vessel, the reservoir, and the outlet collectively configured such that when the frangible vessel is broken, a level of the dinitrogen tetroxide in the reservoir does not reach the outlet regardless of an orientation of the vessel.

Additional advantages will be set forth in part in the description that follows, and in part will be obvious from the description or can be learned by practice of the aspects described below. The advantages described below will be realized and attained by means of the chemical compositions, methods, and combinations thereof, particularly pointed out in the appended claims. It is to be understood that both the foregoing general description and the following detailed description are exemplary and explanatory only and is not restrictive of the invention, as claimed.

### BRIEF DESCRIPTION OF THE DRAWINGS

FIG. 1 is a schematic illustration of a console according to one aspect.

FIG. 2 is a flow diagram of a method for producing nitric oxide according to one aspect.

FIG. 3 is a schematic illustration of a cassette, according to one aspect.

FIG. 4 is a perspective view of a cassette, according to one aspect.

FIG. 5 is a cross-sectional view of the cassette illustrated in FIG. 4.

FIG. 6 is a top view of the cassette illustrated in FIGS. 4 and 5.

FIG. 7 is a perspective view of a liquid vessel assembly, according to one aspect.

FIG. 8 is a cross-sectional view of liquid vessel assembly illustrated in FIG. 7.

FIG. 9 is another cross-sectional view of liquid vessel assembly illustrated in FIG. 7.

FIGS. 10A-10G are perspective views of liquid vessel assembly illustrated in FIG. 7 in different orientations, according to one aspect.

FIG. 11A is an exploded view of a cartridge assembly, according to one aspect.

FIG. 11B is a perspective view of the cartridge assembly of FIG. 11A.

FIG. 12 depicts experimental data showing a positive correlation between cartridge media water content and nitrogen dioxide conversion capacity.

FIG. 13A depicts an exemplary schematic of a system according to one aspect.

FIG. 13B depicts a flow diagram of an exemplary system operation.

FIG. 14 depicts an exemplary setup according to one aspect.

The accompanying figures, which are incorporated in and constitute a part of this specification, illustrate several aspects described below.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

As used herein, the terms “optional” or “optionally” mean that the subsequently described event or circumstance can or cannot occur and that the description includes instances where said event or circumstance occurs and instances where it does not.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used in the description and the appended claims, the singular forms “a,” “an,” and “the” include plural referents unless the context clearly dictates otherwise. Thus, for example, a reference to “a cassette” includes not only one but also two or more such cassettes and a reference to “a console” includes not only one but also two or more such consoles and the like.

Throughout the description and claims of this specification, the word “comprise” and other forms of the word, such as “comprising” and “comprises,” are open, non-limiting terms and mean “including but not limited to,” and are not intended to exclude, for example, other additives, segments, integers, or steps. Furthermore, it is to be understood that the terms “comprise,” “comprising,” and “comprises” as they relate to various aspects, elements, and features of the disclosed invention also include the more limited aspects of “consisting essentially of” and “consisting of.”

Unless defined otherwise, all technical and scientific terms used herein have the same meaning as commonly understood by one of ordinary skill in the art to which this invention belongs. In this specification and in the claims which follow, reference will be made to a number of terms that shall be defined herein.

For the terms “for example” and “such as” and grammatical equivalences thereof, the phrase “and without limitation” is understood to follow unless explicitly stated otherwise. It is further understood that these phrases are used for explanatory purposes only. It is further understood that the term “exemplary,” as used herein, means “an example of” and is not intended to convey an indication of a preferred or ideal aspect.

The expressions “ambient temperature” and “room temperature” as used herein are understood in the art and refer generally to a temperature from about 20° C. to about 35° C.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values, inclusive of the recited values, may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

In still further aspects, when the specific values are disclosed between two end values, it is understood that these end values can also be included.

In still further aspects, when the range is given and exemplary values are provided, it is understood that any ranges can be formed between any exemplary values within the broadest range.

References in the specification and concluding claims to parts by weight of a particular element or component in a composition denote the weight relationship between the element or component and any other elements or components in the composition or article for which a part by weight is expressed. Thus, in a mixture containing 2 parts by weight of component X and 5 parts by weight, components Y, X, and Y are present at a weight ratio of 2:5 and are present in such a ratio regardless of whether additional components are contained in the mixture.

A weight percent (wt. %) of a component, unless specifically stated to the contrary, is based on the total weight of the formulation or composition in which the component is included.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

As used herein, the term “and/or” includes any and all combinations of one or more of the associated listed items.

It will be understood that although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example aspects.

As used herein, the term “substantially” means that the subsequently described event or circumstance completely occurs or that the subsequently described event or circumstance generally, typically, or approximately occurs.

Still further, the term “substantially” can, in some aspects, refer to at least about 90%, at least about 95%, at least about 99%, or about 100% of the stated property, component, composition, or other condition for which substantially is used to characterize or otherwise quantify an amount.

In other aspects, as used herein, the term “substantially free,” when used in the context of a composition or component of a composition that is substantially absent, is intended to refer to an amount that is then about 1% by weight, e.g., less than about 0.5% by weight, less than about 0.1% by weight, less than about 0.05% by weight, or less than about 0.01% by weight of the stated material, based on the total weight of the composition.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

Some aspects described herein relate to methods. It should be understood that such methods can be computer-implemented. That is, where the method or other events are described herein, it should be understood that they may be performed by a computing device having a processor and a memory. Memory of a computing device is also referred to as a non-transitory computer-readable medium, which can include instructions or computer code for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also referred to as code) may be those designed and constructed for a specific purpose or purpose. Examples of non-transitory computer-readable media include, but are not limited to magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules, Read-Only Memory (ROM), Random-Access Memory (RAM) and/or the like. One or more processors can be communicatively coupled to the memory and operable to execute the code stored on the non-transitory processor-readable medium. Examples of processors include general purpose processors (e.g., CPUs), Graphical Processing Units, Field Programmable Gate Arrays (FPGAs), Application Specific Integrated Circuits (ASICs), Digital Signal Processor (DSPs), Programmable Logic Devices (PLDs), and the like. Examples of computer code include but are not limited to, micro-code or micro-instructions, machine instructions, such as produced by a compiler, code used to produce a web service, and files containing higher-level instructions that are executed by a computer using an interpreter. For example, aspects may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include but are not limited to, control signals, encrypted code, and compressed code.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

The present invention may be understood more readily by reference to the following detailed description of various aspects of the invention and the examples included therein and to the Figures and their previous and following description.

### Device

In certain aspects, disclosed herein is a console. It is understood that the console disclosed herein can be used for medical purposes and, more specifically, for delivering nitric oxide to a subject. In aspects disclosed herein, the console can comprise various compartments and elements configured to deliver the desired amount of nitric oxide to the subject.

In one aspect, disclosed herein is a console comprising: at least one air inlet; a first receptacle configured to host a first source of nitric oxide and a second receptacle configured to host a second source of nitric oxide. In other words, the console disclosed herein is configured to host at least two sources that can provide the desired amount of nitric oxide.

In still further aspects, the console comprises a controller configured to selectively couple the at least one air inlet to one of the first receptacles or the second receptacle to deliver the nitric oxide from the first or the second source. In still further aspects, the console comprises an outlet coupled to the first and/or second receptacle and configured to deliver nitric oxide to a subject.

It is understood that the source of nitric oxide can be any source known in the art. For example, the source of nitric oxide can be nitric oxide pressurized in a tank. In such aspects, the receptacle can be adjusted to either host the tank itself or host nitric oxide delivery elements that are in communication with the tank and provide the desired amount of nitric oxide as needed.

Yet, in still further aspects, the nitric oxide can be formed in-situ. In such aspects, the first source and the second source can comprise materials configured to form nitric oxide if desired. For example, and without limitations, the first and the second sources of nitric oxide can comprise air or a nitrite solution and an electrical arrangement, allowing the formation of nitric oxide through a spark.

In some aspects, the first and the second source of nitric oxide can comprise nitrogen dioxide. Yet in other exemplary and unlimiting aspects, the first and the second source of nitric oxide can comprise a liquid dinitrogen tetroxide. In this exemplary aspect, the liquid dinitrogen tetroxide can be contained in a reservoir (for example, a first reservoir and a second reservoir, respectively, for the first and the second source of nitric oxide). The reservoir(s) can be positioned within a cassette(s). The liquid dinitrogen dioxide can be a source of gaseous nitrogen dioxide that can be converted to nitric oxide in the cassette. In still further aspects, the first source of nitric oxide comprises a first cassette configured to form nitric oxide and/or wherein the second source of nitric oxide comprises a second cassette configured to generate nitric oxide. In still further aspects, when the at least one air inlet Is coupled to the first receptacle and the first source of nitric oxide is substantially depleted, the controller is configured to automatically switch the coupling of at least one air inlet from the first receptacle to the second receptacle thereby delivering nitric oxide from the second source.

In yet still further aspects, the controller is configured to monitor a fill state of the first and/or second nitric oxide source.

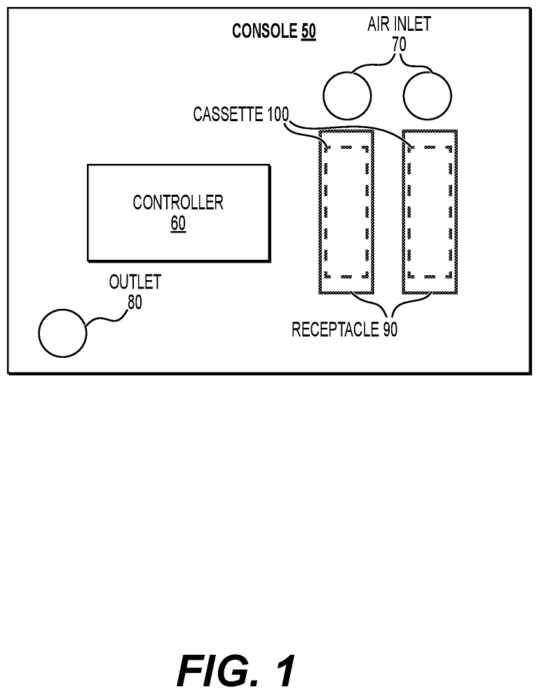

Exemplary and unlimiting console is shown in FIG. 1. FIG. 1 is a schematic illustration of a console 50, according to an aspect. Console 50 is an on-demand delivery system for nitric oxide. As shown in FIG. 1, console 50 includes controller 60, one or more air inlet(s) 70, air outlet 80, and one or more receptacle(s) 90 configured to receive cassette(s) 100.

Controller 60 in the console 50 can be operable to cause nitric oxide to be delivered at a controlled flow rate and/or concentration. In such aspects, the disclosed herein controller is configured to control a rate of nitric oxide delivered to a subject. Yet in still further aspects, the controller is also configured to deliver nitric oxide at the desired concentration.

For example, the controller 60 can contain one or more processors, memory, and/or control circuits operable to, for example, selectively activate individual cassettes 100 and/or control flow rates through the console 50. In such aspects, the controller is configured to control a rate of nitric oxide generation in the first cassette and/or second cassette. For example, the controller 60 can be operable to control one or more pumps to cause air to be drawn through one or more air inlets 70, selectively control flow through one or more cassette(s) 100, control temperature of liquid dinitrogen tetroxide within a cassette(s) 100, control a rate at which nitrogen dioxide is produced within a cassette(s) 100, control a rate at which nitric oxide is produced within a cassette(s) 100, control a concentration and/or flow rate at which nitric oxide leaves a cassette(s) 100, control a concentration and/or flow rate at which a nitric oxide-containing gas leaves the console 50 (e.g., through one or more air outlet(s) 80) for delivery to a patient, and/or so forth. The controller 60 can include one or more user interfaces or controls such that nitric oxide can be delivered on demand and/or such that a user can interface with the controller 60 to select, for example, flow rate and/or nitric oxide flow concentration.

A cassette 100 can be configured to be inserted into each of one or more receptacles 90, and upon activation, produce nitric oxide. Each cassette 100 can be a single-use, disposable component that stores liquid dinitrogen tetroxide (N₂O₄). N₂O₄ can be converted into gaseous nitrogen dioxide (NO₂), and each cassette can be activated to convert NO₂ into nitric oxide (NO).

In some aspects, the console 50 can be designed and/or configured to contain more than one receptacle 90 and more than one cassette 100. Each receptacle 90 is configured to receive one cassette 100. The controller 60 can be capable of automatically changing from a first receptacle/cassette to a second receptacle/cassette when the first cassette nears depletion. Similarly stated, the controller 60 can be operable to source nitric oxide selectively from any cassette 100, monitor a fill-state of each cassette 100, and/or transition from sourcing nitrogen dioxide from a first cassette to a second cassette, for example, when the first cassette nears an empty state. This simplifies the usability of delivering the console 50 by eliminating operator steps required to control the transition between two separate consoles when a first cassette nears depletion and extends the time of nitric oxide delivery.

In some aspects, the console 50 can be designed and/or configured to contain only one air inlet 70. When the console 50 contains more than one receptacle(s) 90 and more than one cassette(s) 100, the controller 60 can be capable of selectively coupling the air inlet 70 to one of the receptacle(s) 90 such that the console 50 can automatically transition from producing nitric oxide using one of the cassette(s) 100. For example, the controller 60 can be capable of automatically changing from coupling the air inlet 70 to a first receptacle/cassette to coupling the air inlet 70 to a second receptacle/cassette when the first cassette nears depletion.

In some aspects, the console 50 can be designed and/or configured to contain more than one air inlet(s) 70. For example, the console can comprise two or more air inlets, such that the first receptacle and the second receptacle are separately coupled to at least one air inlet.

In such aspects, each air inlet 70 is separately coupled to one of the more than one receptacle(s) 90 and one of the more than one cassette(s) 100. The controller 60 can be capable of selectively activating the air inlet 70 that is coupled with one of the receptacle and one of the cassettes, such that the console 50 can automatically transition from producing nitric oxide using one of the cassette(s) 100. For example, the controller 60 can be capable of automatically changing from activating a first air inlet 70 coupled to a first receptacle/cassette to activating a second air inlet 70 coupled to a second receptacle/cassette when the first cassette nears depletion. In other words, the controller is configured to selectively activate the at least one air inlet in the first receptacle or the second receptacle based on a fill state of the first and/or second nitric oxide source. In exemplary aspects disclosed herein, the controller is configured to detect the fill state of the liquid dinitrogen tetroxide that is used as a source of nitric oxide.

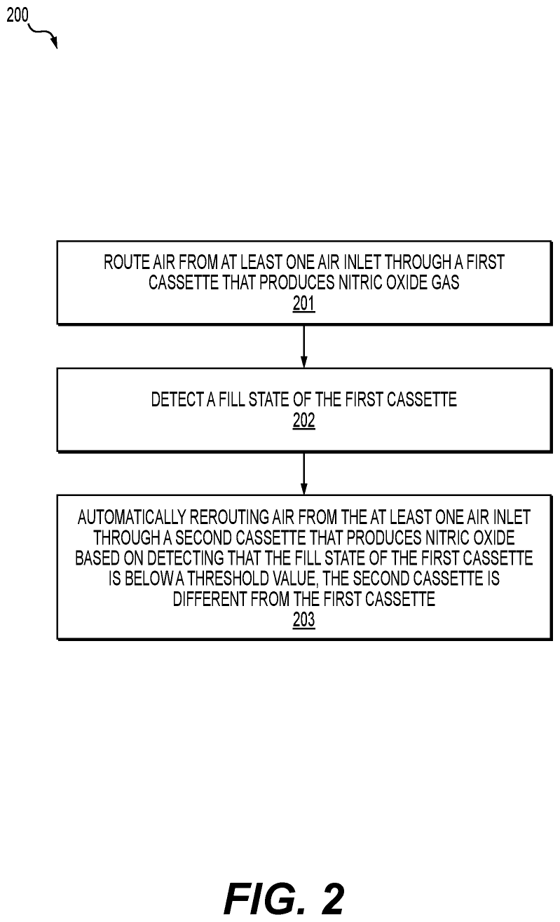

FIG. 2 is a flow diagram of a method for producing nitric oxide, according to an exemplary aspect disclosed herein. The method 200 of FIG. 2 can be implemented, for example, using the controller 60 of the console 50 in FIG. 1.

As shown in FIG. 2, the method 200 begins with routing air from at least one air inlet (e.g., air inlet(s) 70 in FIG. 1) through a first cassette (e.g., cassette(s) 100 in FIG. 1) that produces nitric oxide gas, at 201. For example, a controller (e.g., controller 60 in FIG. 1) can control an air inlet (e.g., an air inlet 70 in FIG. 1) to route air through a first cassette (e.g., cassette(s) 100 in FIG. 1). The controller can further be operable to control a rate at which nitric oxide is generated by the cassette, for example, by controlling a temperature of a reservoir containing dinitrogen tetroxide and/or a concentration at which nitric oxide is delivered, for example, by controlling a flow rate of a carrier gas from air inlet to air outlet.

At 202, the method continues to detect a dinitrogen tetroxide fill state of the first cassette. The controller (e.g., controller 60 in FIG. 1) monitors and detects a dinitrogen tetroxide fill state of the first cassette (e.g., cassette(s) 100 in FIG. 1), while the first cassette produces nitric oxide. The dinitrogen tetroxide fill state is used to determine an appropriate time when the first cassette approaches depletion to stop dosing using the first cassette and initiate dosing with a second cassette. This method improves the utilization of cassette dinitrogen tetroxide source material to reduce the remaining material (e.g., dinitrogen tetroxide, nitrogen dioxide) at the end of dosing for each cassette while maintaining continuous nitric oxide dose delivery. The dinitrogen tetroxide fill state can be detected, for example, by monitoring a temperature and/or pressure of a liquid vessel assembly (e.g., liquid vessel assembly 3000 discussed in further detail below). As a quantity of dinitrogen tetroxide in a liquid vessel assembly decreases (i.e., nears depletion), the temperature required to achieve an internal pressure to produce a given quantity/concentration of nitric oxide can increase. A feedback control mechanism can measure the quantity/concentration of nitric oxide produced by a cassette and adjust the temperature set point for the liquid vessel assembly to maintain a set level and/or produce a target quantity/concentration. The controller can determine that a cassette is nearing depletion based on the temperature and/or pressure within the liquid vessel assembly and/or the rate at which the cassette is producing nitric oxide.

At 203, the method continues to automatically re-route air from the at least one air inlet (e.g., air inlet(s) 70 in FIG. 1) through a second cassette (e.g., cassette(s) 100 in FIG. 1) that produces nitric oxide based on detecting that the fill state of the first cassette is below a threshold value. For example, when the controller (e.g., controller 60 in FIG. 1) detects that the fill state of the first cassette (e.g., cassette(s) 100 in FIG. 1) is below a predefined threshold value, the controller automatically re-route the air from air inlet (e.g., air inlet(s) 70 in FIG. 1) through a second cassette (e.g., cassette(s) 100 in FIG. 1) to ensure the continuity of the airflow produced. The first cassette is different from the second cassette. In some instances, at 203, the method can further include activating the second cassette, for example, by breaking or rupturing a vessel containing liquid dinitrogen tetroxide (e.g., an ampule).

Control algorithms for producing nitric oxide can also be switched from controlling the first cassette to controlling the second cassette. In some aspects, the switch can be step-wise. In still further aspects, the controller is configured to gradually increase a concentration of nitric oxide from the second source while still delivering nitric oxide from the first source. For example, the second cassette can be ramped up (e.g., by increasing a temperature of a liquid vessel in the second cassette to produce a target level of nitric oxide), while the first cassette retains responsibility for supplying nitric oxide via the air outlet. During the ramp-up phase, nitric oxide produced from the second cassette can be vented into room air or inerted. Once the second cassette has reached the target level, a valve can disconnect the first cassette from the air outlet and connect the second cassette to the air outlet. Any residual nitric oxide produced by the disconnected first cassette can be vented into room air and/or inerted. In other aspects, the switch from the first cassette to the second cassette can be gradual.

For example, control algorithms can gradually increase the concentration of nitric oxide produced by the second cassette (e.g., by increasing the temperature of a liquid vessel of the second cassette) while decreasing the concentration of nitric oxide produced by the first cassette (e.g., by reducing the temperature of a liquid vessel of the first cassette). During the transition period, both the first cassette and second cassette can be coupled to an air outlet and contribute to supplying nitric oxide. In other words, also disclosed are aspects, wherein, at least once, the at least one air inlet is coupled to the first receptacle and the second receptacle simultaneously.

In some aspects, for example, the system only contains one air inlet (e.g., air inlet(s) 70 in FIG. 1) while the system contains more than one cassette (e.g., cassette(s) 100 in FIG. 1). The controller (e.g., controller 60 in FIG. 1) can be capable of routing the air from the air inlet through a first cassette (e.g., cassette(s) 100 in FIG. 1) that produces nitric oxide gas by coupling the air inlet to the first cassette. The controller can monitor and detect a fill state of the first cassette while the first cassette is producing nitric oxide. When the controller detects that the fill state of the first cassette is below a threshold value, the controller can automatically re-route the air from the air inlet through a second cassette (e.g., cassette(s) 100 in FIG. 1) that produces nitric oxide gas by coupling the air inlet to the second cassette to ensure the continuity of airflow.

In some aspects, for example, the system contains more than one air inlet (e.g., air inlet(s) 70 in FIG. 1) while the system contains more than one cassette (e.g., cassette(s) 100 in FIG. 1). Each air inlet is separately coupled to one of the more than one cassettes. The controller (e.g., controller 60 in FIG. 1) can be capable of routing the air from a first air inlet (e.g., air inlet(s) 70 in FIG. 1) through a first cassette (e.g., cassette(s) 100 in FIG. 1) that produces nitric oxide gas by activating the first air inlet. The controller can monitor and detect a fill state of the first cassette while the first cassette is producing nitric oxide. When the controller detects that the fill state of the first cassette is below a threshold value, the controller can automatically re-route the air from the second air inlet through a second cassette (e.g., cassette(s) 100 in FIG. 1) that produces nitric oxide gas by switching to and activating the second air inlet to ensure the continuity of airflow.

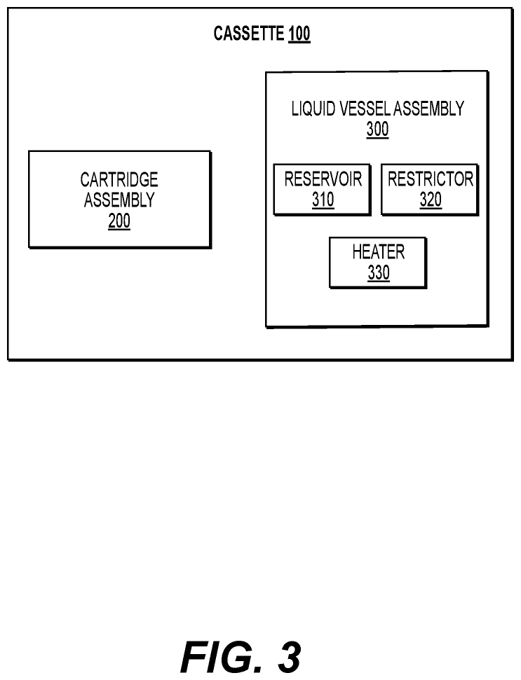

FIG. 3 is a schematic illustration of a cassette 100, according to an aspect. U.S. Pat. No. 8,887,720, the disclosure of which is hereby incorporated by reference in its entirety, describes a known reservoir assembly and cartridge that is suitable for use with cassette 100. The cassette 100 includes a cartridge assembly 200 and a liquid vessel (LV) assembly 300. The LV assembly 300 includes a reservoir 310, a restrictor 320, and a heater 330. The reservoir 310 can contain liquid dinitrogen tetroxide. Upon activation (e.g., by the console 50), heater 330 increases the temperature of the liquid dinitrogen tetroxide, which produces nitrogen dioxide. The nitrogen dioxide can exit the reservoir 310 through restrictor 320. Together, the heater 330 and the restrictor 320 can provide for a controlled release of nitrogen dioxide. After passing through the restrictor, the nitrogen dioxide can flow to the cartridge assembly 200, which can be operable to convert nitrogen dioxide to nitric oxide, for example, through a chemical reaction. The chemical reaction, for example, can be a reaction of nitrogen dioxide with an antioxidant coated on a surface-active material that retains water. In some implementations, the antioxidant can be ascorbic acid, and the surface-active material can be silica gel. Yet, in other aspects, nitric oxide can be formed from nitrogen dioxide in the presence of an amount of water without the presence of other antioxidants. The generated nitric oxide is then delivered to the patient through the console 50.

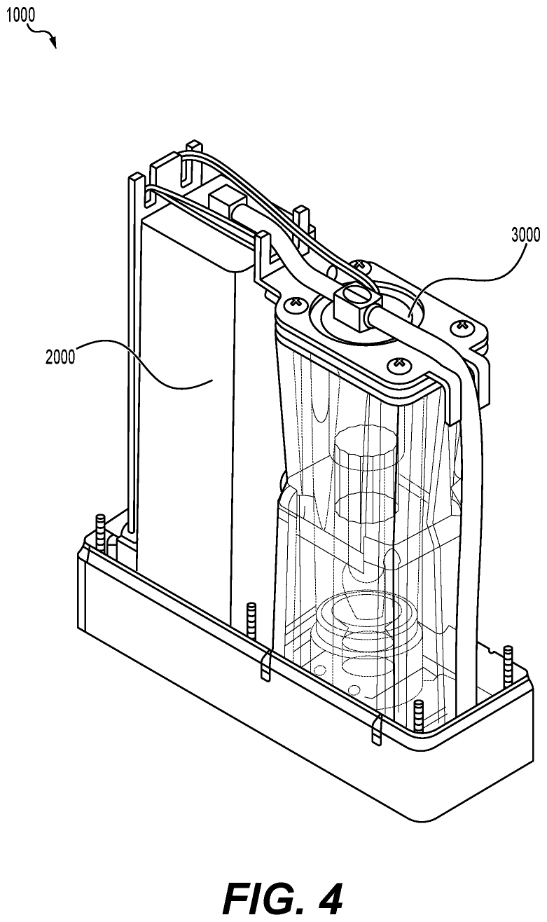

FIG. 4 is a perspective view of a cassette 1000, according to an aspect. The cassette 1000 includes a cartridge assembly 2000 and a liquid vessel (LV) assembly 3000. The LV assembly 3000 can contain liquid dinitrogen tetroxide. Upon activation, the liquid dinitrogen tetroxide can be heated to produce nitrogen dioxide. The nitrogen dioxide can flow into the cartridge assembly 2000 for further conversion. The cartridge assembly 2000 can be operable to convert nitrogen dioxide to nitric oxide, for example, through a chemical reaction. The chemical reaction can be a reaction of nitrogen dioxide with an antioxidant coated on a surface-active material that retains water. In some implementations, the antioxidant can be ascorbic acid, and the surface-active material can be silica gel.

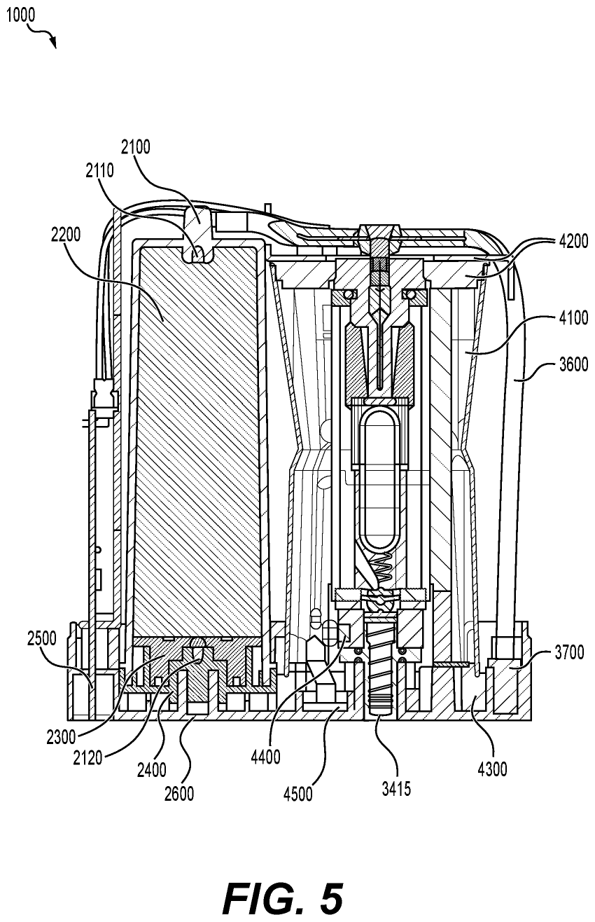

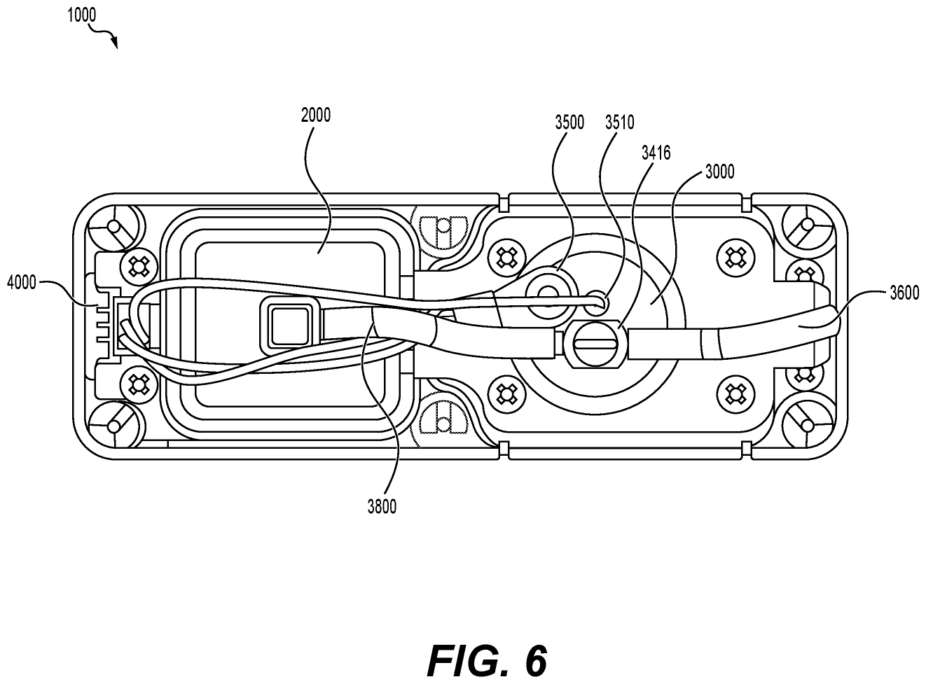

In other aspects, the antioxidant can be water. The generated nitric oxide is then delivered to the patient through a console. FIG. 5 is a cross-sectional view of the cassette 1000 illustrated in FIG. 4. FIG. 6 is a top view of the cassette 1000 illustrated in FIGS. 4 and 5. As shown in FIG. 5, the cartridge assembly 2000 includes a cartridge housing 2100, cartridge inlet frit 2110, cartridge media 2200, cartridge edge connector 2500, compression stopper 2300, cartridge outlet frit 2120, cartridge housing lid 2400, and output gas port 2600. The cartridge assembly 2000 is configured to convert nitrogen dioxide into nitric oxide through a chemical reaction. The chemical reaction can be a reaction of nitrogen dioxide with an antioxidant coated on a surface-active material that retains water. The cartridge media 2200 contained in the cartridge assembly 2000 can be an antioxidant coated on a surface-active material. In some implementations, the antioxidant can be ascorbic acid, and the surface-active material can be silica gel. Yet, it is understood that water can be used as an antioxidant. In yet still further aspects, other known antioxidants such as Vitamin C, Vitamin E, alpha-tocopherol, gamma-tocopherol, or any combination thereof can be used.

The cartridge inlet frit 2110 and cartridge outlet frit 2120 are configured to retain the cartridge media 2200 inside the cartridge housing 2100 and provide a uniform flow of air flowing into and out of the cartridge through the frits. The compression stopper 2300 is configured to hold the cartridge media 2200 inside the cartridge housing 2100 with compression. The compression stopper 2300 can be manufactured from an elastomeric or any other suitable material that allows the compression stopper 2300 to conform to the cartridge housing 2100 and/or compress cartridge media 2200. Compression stopper 2300 can be chemically non-reactive with the cartridge media 2200, antioxidants, nitrogen dioxide, and/or nitric oxide. The cartridge housing lid 2400 is configured to seal and close the cartridge housing 2100 so that the cartridge media 2200 contained in the cartridge housing 2100 can be protected and separated from room air. The output gas port 2600 is configured to output the produced nitric oxide with a controlled flow rate to be delivered to the patient.

The cassette edge connecter 2500 is configured to electrically and/or communicatively connect the cassette 1000 to a control unit that is capable of controlling the delivery of nitric oxide. As shown in FIG. 6, the control unit can be cassette printed circuit board assembly 4000. In some implementations, the cassette printed circuit board assembly 4000 can be operable to control one or more pumps to cause air to be drawn through the air gas port 3700, selectively control flow through one or more cassette(s) 1000, control temperature of liquid dinitrogen tetroxide within a cassette(s) 1000, control a rate at which nitrogen dioxide is produced within a cassette(s) 1000, control a rate at which nitric oxide is produced within a cassette(s) 1000, control a concentration and/or flow rate at which nitric oxide leaves a cassette(s) 1000 through the output gas port 2600, and/or so forth. In some implementations, the cassette printed circuit board assembly 4000 can be connected to a single heater element and is configured to control the temperature of dinitrogen tetroxide within the cassette 1000. Upon activation, the cassette-printed circuit board assembly 4000 can control a single heater element to heat the temperature at an elevated level. The liquid vessel heater 3510 can be a single cartridge heater, as shown in FIG. 6. The liquid vessel heater 3510 can be thermally coupled to the reservoir by a restrictor body 3210 (as best shown in FIG. 6) inside the liquid vessel assembly 3000. In some implementations, multiple heaters can be used in the liquid vessel assembly 3000 to increase and/or control the temperature of dinitrogen tetroxide.

In some implementations, the cassette-printed circuit board assembly 4000 can receive/send signals from a console (e.g., console 50 in FIG. 1) and implement instructions received from the console. For example, the cassette printed circuit board assembly 4000 can activate the liquid vessel heater 3510 on the liquid vessel assembly (3000) in response to instructions received from the console and/or sending signals from thermistor 3500 back to the console such that the console can monitor the temperature in cassette 1000. In such implementations, the console may control one or more pumps that cause air to be drawn through the air gas port 3700 and flow through one or more cassette(s) 1000.

The temperature is monitored and/or controlled with a single thermistor 3500, as shown in FIG. 6. The thermistor 3500 is attached to the top surface of the liquid vessel assembly 3000, as shown in FIG. 6, via a spade tab hole connection secured with a screw (not shown). Compared to wired heater coil heating elements, the single heater element liquid vessel heater 3510 has improved mechanical strength and durability, which reduces the potential for wire/coil fracture failures. The process to secure the liquid vessel heater 3510 and thermistor 3500 to the liquid vessel assembly 3000 is substantially simplified compared to the process of securing wired heater coil heating elements, as implemented in some known cassettes. In some implementations, multiple thermistors can be used and attached to the liquid vessel assembly 3000 to monitor and/or control the temperature.

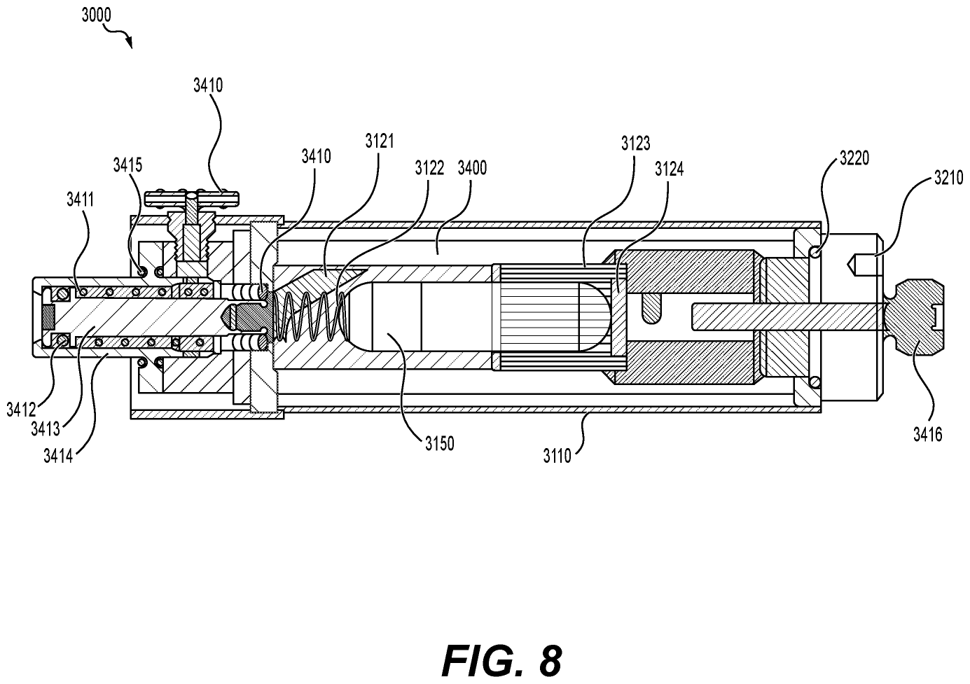

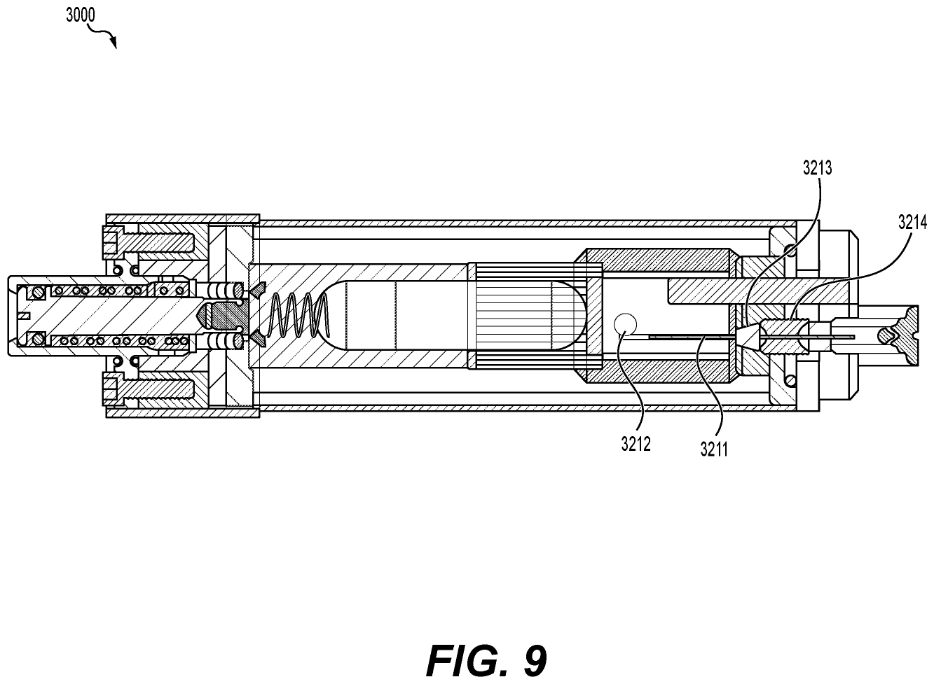

The input air tubing 3600 and input air gas port 3700, as shown in FIG. 5, are configured to guide input air into the cassette 1000. The nitrogen dioxide-containing gas tubing 3800, as shown in FIG. 6, is configured to guide the generated nitrogen dioxide into the cartridge assembly 2000 to be further converted into nitric oxide. The activation pin opening 3415 contains a pin for activation of the cassette 1000, which will be discussed in detail in the descriptions of FIG. 8. The tee fitting 3416, as shown in FIG. 6, is coupled to a restrictor (e.g., capillary restrictor tubing 3211, as shown in FIG. 9) and introduces nitrogen dioxide produced in the liquid vessel assembly into carrier gas (e.g., air) flowing from input air gas port 3700, through input air tubing 3600, and onto the nitrogen dioxide-containing gas tubing 3800 and cartridge assembly 2000.

As shown in FIG. 5, the inerting chamber 4100 is a chamber that is configured to encase the liquid vessel assembly 3000 to prevent accidental nitrogen dioxide and/or dinitrogen tetroxide release from the cassette 1000. The inerting chamber top gasket and clamp 4200 and inerting chamber bottom gasket 4300 are configured to connect the inerting chamber 4100 with the liquid vessel assembly 3000. The inerting chamber top gasket and clamp 4200 and inerting chamber bottom gasket 4300 can be made of elastic materials. In the event, the ampule 3150 is breached in a scenario in which the cassette 1000 is not being used to generate nitric oxide (e.g., in transit, if dropped, etc.), the dinitrogen tetroxide contained in the cassette 1000 and/or nitrogen dioxide can be vented into the inerting chamber 4100 through the inerting vent path 4400. Color indicator 4500 can change color to alert the user that nitrogen dioxide has been discharged into the inerting chamber 4100.

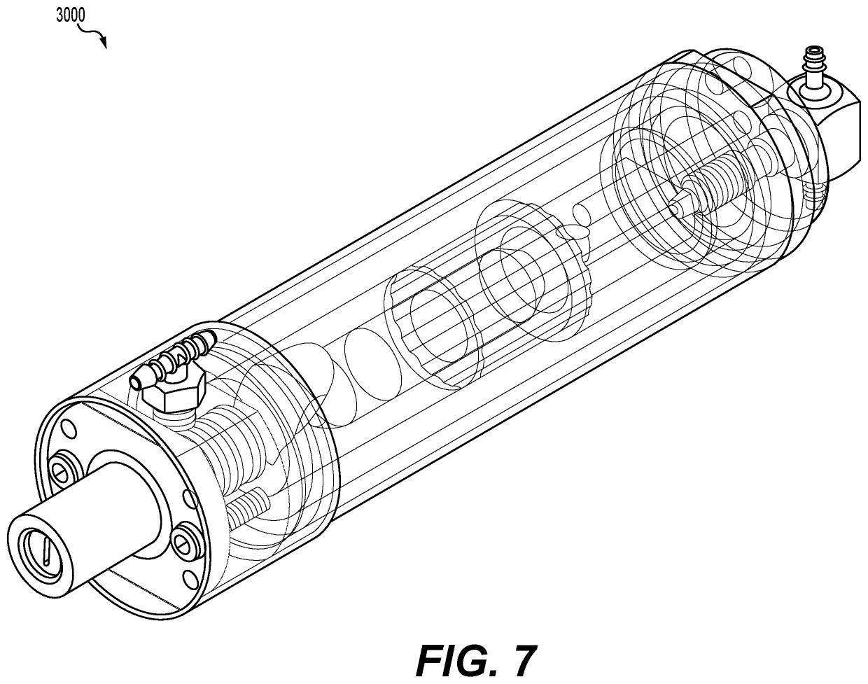

FIG. 7 is a perspective view of a liquid vessel assembly 3000, according to an aspect. FIG. 8 is a cross-sectional view of liquid vessel assembly 3000 illustrated in FIG. 7. FIG. 9 is another cross-sectional view of liquid vessel assembly 3000 illustrated in FIG. 7. As mentioned above, the liquid vessel (LV) assembly 3000 is configured to store liquid dinitrogen tetroxide. The liquid dinitrogen tetroxide can be heated to produce nitrogen dioxide.

Still further disclosed herein is an apparatus comprising: a frangible vessel containing dinitrogen tetroxide; a reservoir, the frangible vessel disposed within the reservoir, the reservoir configured to contain the dinitrogen tetroxide when the frangible vessel is broken; and an outlet, the outlet disposed above a wall of the reservoir, the frangible vessel, the reservoir, and the outlet collectively configured such that when the frangible vessel is broken, a level of the dinitrogen tetroxide in the reservoir does not reach the outlet regardless of an orientation of the vessel.

Such an exemplary aspect is also shown in FIG. 8. As shown in FIG. 8, the LV assembly 3000 includes an ampule 3150 (e.g., a frangible vessel). The ampule 3150 is configured to store liquid dinitrogen tetroxide. The LV assembly 3000 includes an activation mechanism that can break the ampule 3150 to release the liquid dinitrogen tetroxide into a reservoir within the LV assembly 3000. The activation mechanism includes activation wedge 3121, activation wedge spring 3122, activation sleeve 3123, ePTFE pad 3124, activation sleeve (bottom) 3414, activation pin 4313, activation pin O-ring 3412, activation pin spring 3411, and LV housing 3400.

Prior to activation/breakage, the ampule 3150 is secured in position laterally with an expanded PTFE foam pad (ePTFE pad 3124) under the bottom end of the ampule 3150 and a low contact force conical spring (activation wedge spring 3122) on the activation top end of the ampule 3150. The ePTFE pad 3124 and the activation wedge spring 3122 dampen mechanical shock on the ends of the ampule 3150. The ampule 3150 is also supported around its circumference for the bottom half of its length using an aluminum sleeve (activation sleeve 3123), which has a minimal clearance between the inner diameter of the activation sleeve 3123 and the outer diameter of the ampule 3150 to minimize potential shock from side impact. These features improve the mechanical durability of the ampule 3150.

The breakage/activation of the ampule 3150 is achieved using the activation wedge 3121. The leading edge of the activation wedge 3121 applies lateral force to the unsupported free end of the ampule 3150 when it makes contact during actuation. The lateral force flexes the ampule 3150 against the cylindrical activation sleeve 3123, which maintains the ampule 3150 position with concentric alignment with the LVM housing 3400 and supports it for approximately half the ampule 3150 length. Lateral flexing forces create strain at the ampule 3150 wall at the mid-point of the ampule 3150 length, which fractures the ampule 3150. During actuation and prior to breakage, the larger diameter of the inside of the LVM housing 3400 forms a gas seal with the U-cup seals 3410. Prior to and after use, the activation pin 3413 is in the retracted position. In the retracted position, the smaller diameter portion of the inside of the LVM housing 3400 maintains an open venting gas path (inerting vent path 4400 as shown in FIG. 5) between the inside of the LV assembly 3000 and the inerting chamber 4100 (as shown in FIG. 5). The inerting chamber 4100 can contain absorbent media such as soda lime chemical absorbent media, which can be suitable to neutralize dinitrogen tetroxide and/or nitrogen dioxide, preventing the release of toxic gasses from escaping the LV assembly.

The activation sleeve (bottom) 3414, activation pin 3413, activation pin O-ring 3412, and activation pin spring 3411 are also part of the activation mechanism. The activation sleeve 3414 is configured to protect the activation pin 3413 and hold the activation pin 3413 in place prior to or after the activation. The activation pin 3413 and the activation pin spring 3411 are disposed in the activation pin opening 3415, as shown in FIG. 5. The activation pin 3413 and the activation pin spring 3411 are configured to activate the LV assembly 3000 by applying a constant force to the activation wedge 3121 and activation wedge spring 3122. The activation pin O-ring 3412 is configured to seal the LV assembly 3000 and prevent liquid/gas from leaking.

After activation, the liquid dinitrogen tetroxide is released into from the ampule 3100 and into a reservoir. Further, activation can seal the inerting vent path 4400. The reservoir can be heated by a heater to produce nitrogen dioxide as well as to control the pressure within the reservoir. The nitrogen dioxide can exit the LV assembly 3000 through a restrictor. By controlling the pressure (which is a function of temperature), the nitrogen dioxide can be released through the restrictor at a controlled rate. After passing through the restrictor, the nitrogen dioxide can flow with a carrier gas supplied by the console to the cartridge assembly 2000, which can be operable to convert nitrogen dioxide to nitric oxide. As shown in FIG. 8 and FIG. 9, the LV assembly 3000 contains a restrictor body 3210 and a capillary restrictor tubing 3211. A single liquid vessel heater 3510 (as shown in FIG. 6) is loaded into a hole in the restrictor body 3210 in proximity to the capillary restrictor tubing 3211 for heating the liquid dinitrogen tetroxide to produce nitrogen dioxide. The proximity of the liquid vessel heater 3510 to the capillary restrictor tubing 3211 is positioned to keep the restrictor body 3210 temperature remains elevated compared to the LV assembly 3000 temperature, which reduces the likelihood of liquid condensation and clogging of the capillary restrictor tubing 3211. In some implementations, multiple heaters can be used in the LV assembly 3000 to heat the temperature of dinitrogen tetroxide.

The body of the liquid vessel assembly 3000 can be manufactured using aluminum, which reduces machining cost and material cost. In fact, the majority of the LV assembly 3000 components can be manufactured using aluminum for the same reason. Aluminum provides high thermal conductivity and heat transfer for the heat generated at the restrictor body 3210 end of the LV assembly 3000. It may be desirable for the restrictor body 3210 to be constructed of a material with higher thermal capacity, such as stainless steel, than the body of the liquid vessel assembly 3000, which can provide comparatively better heat retention during cooling (e.g., during periods in which the cartridge heater is off), which can keep the capillary restrictor tubing 3211 warmer than the reservoir, further inhibiting condensation.

As mentioned above, the temperature can be monitored and/or controlled using a single thermistor 3500 attached to the restrictor body 3210 top surface via a spade tab hole connection secured with a screw. The single thermistor 3500 has improved mechanical strength and durability, which reduces the potential for wire fracture failures. In some implementations, multiple thermistors can be used and attached to the liquid vessel assembly 3000 to monitor the temperature.

The LV assembly 3000 also includes an insulating polymer sleeve 3110 for insulating the LV assembly 3000 and reducing heat loss. The insulation sleeve 3110 is applied around the outer surface of the LV assembly 3000 during assembly. This insulation sleeve 3110 creates a small air gap between the outer surface of the LV assembly 3000 and the surrounding absorbent media in the inerting chamber 4100 (as shown in FIG. 5). This air gap provides a thermal insulating layer which reduces the rate of heat loss to the surrounding media and reduces the heating time to a target temperature by approximately 2 times compared to heating with the same input power and no insulation sleeve. The tee fitting 3416 is the same as shown in FIG. 6 and is configured to introduce nitrogen dioxide produced in the reservoir into the carrier gas (e.g., air or oxygen). The ferrule seal 3213 and the vented set screw 3214 are configured to join the pipes (e.g., capillary restrictor tubing 3211 and the tee fitting 3416) and allow the release of nitric oxide flow.

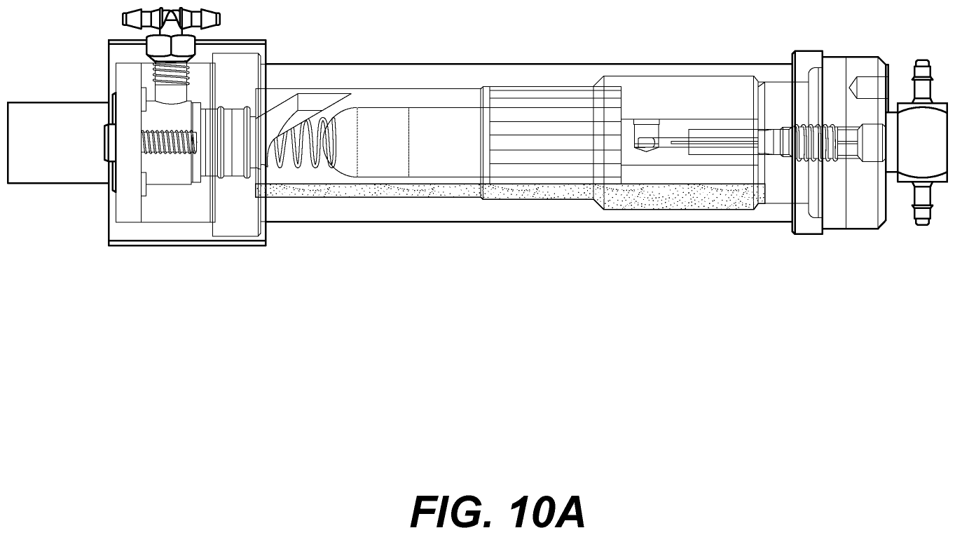

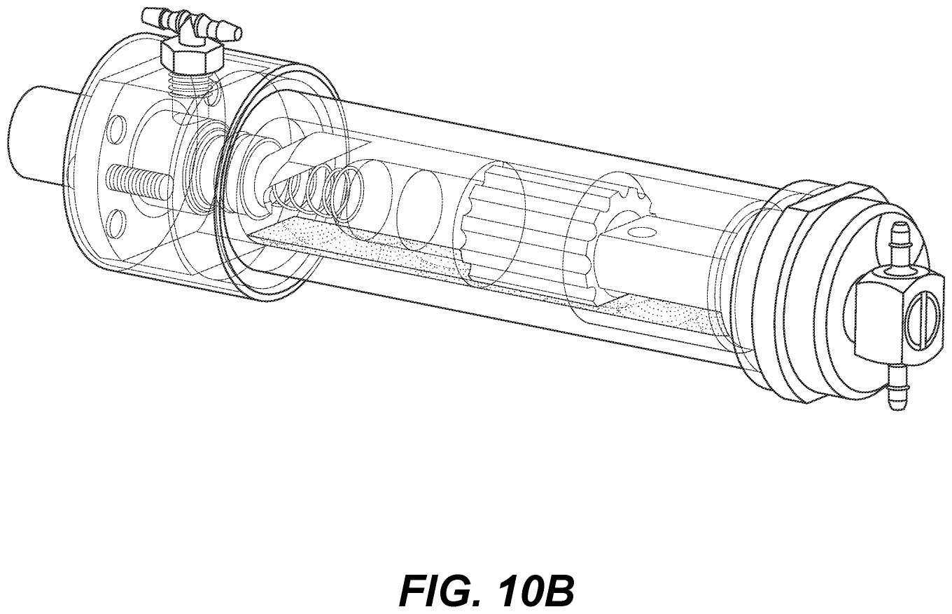

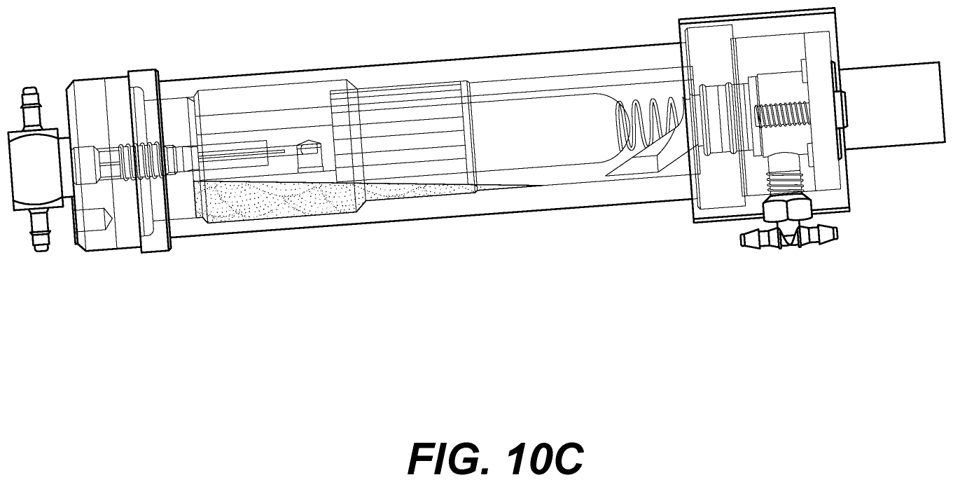

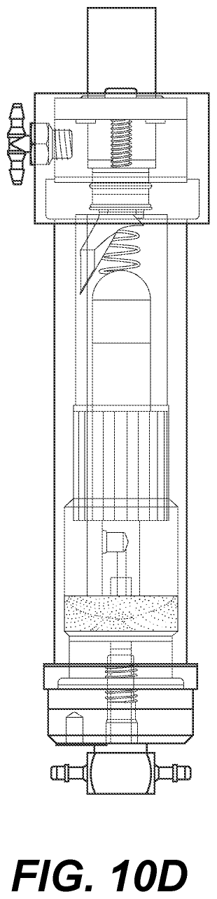

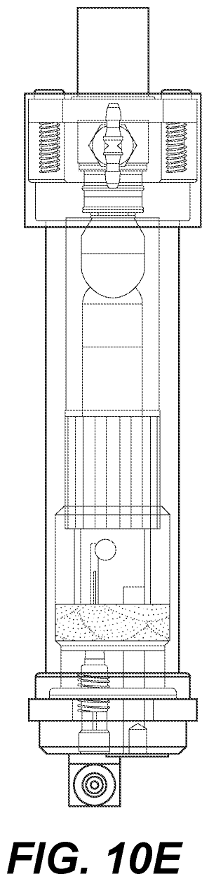

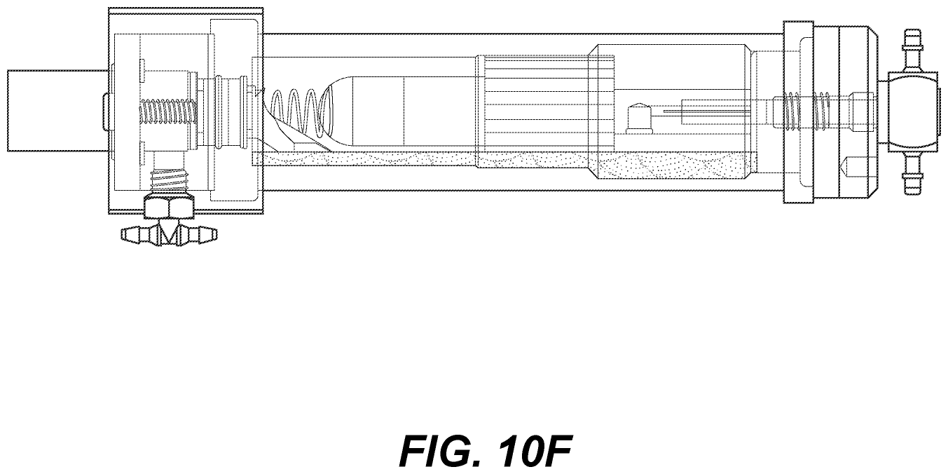

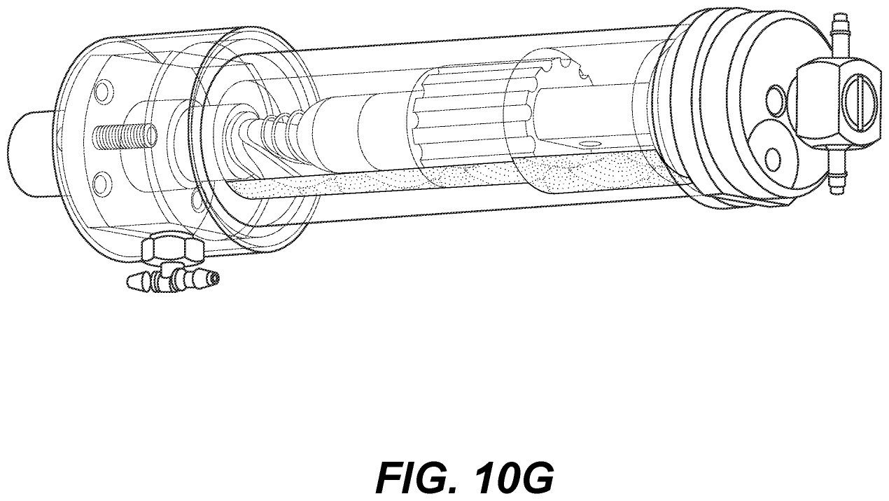

In still further aspects, the apparatus comprises a separator disposed in the outlet and positioned above dinitrogen tetroxide regardless of the orientation of the vessel or external vibration applied to the vessel. For example, the LV assembly 3000 includes a gas intake frit (or a separator) 3212 coupled to the capillary restrictor tubing 3211 and tee fitting 3416. The gas intake frit 3212, capillary restrictor tubing 3211 and tee fitting 3416 collectively define a flow path through which nitrogen dioxide exits the LV assembly 3000. As seen best in FIGS. 9 and 10B, the gas intake frit 3212 is disposed on a pedestal that is coaxial with and disposed within the LV assembly 3000. In this way, the gas intake frit 3212 can be positioned above the liquid level of dinitrogen tetroxide in the reservoir, regardless of the orientation of the LV assembly 3000. This feature improves the performance of the LV assembly 3000 for a wider range of use conditions, for example, when the patient or the cassette may be moved during use, such as inpatient transport or other ambulatory/portable use applications. For example, the location of the gas intake frit 3212 allows the liquid vessel assembly to be operated in any orientation without liquid contacting the gas intake frit 3212, which can clog the gas path and inhibit nitrogen dioxide from exiting the liquid vessel assembly 3000. This can allow the cassette 1000 to be disposed in a console that may be carried or in a vehicle, such as an ambulance or helicopter. Such a console can be used without the risk of nitric oxide interruption due to changes in orientation or vibration.

FIGS. 10A-10G are side views of liquid vessel assembly illustrated in FIG. 7 in different orientations. As mentioned above, the gas intake frit 3212 is positioned so the liquid fill height does not reach the frit intake location with any orientation of the LV assembly 3000. A few examples of liquid levels in extreme orientation conditions are shown in FIGS. 10A-10E. FIG. 10A and FIG. 10B shows liquid dinitrogen tetroxide fill level in an upright orientation at room temperature. FIG. 10C shows liquid dinitrogen tetroxide fill level in a 5-degree tilt orientation at room temperature. FIG. 10D and FIG. 10E shows liquid dinitrogen tetroxide fill level in a 90-degree rotate orientation at room temperature. FIG. 10F and FIG. 10G shows liquid dinitrogen tetroxide fill level in an upside-down orientation at room temperature. As shown in FIGS. 10A-10G, in any of the extreme orientation conditions of LV assembly 3000, the liquid fill height does not reach the frit intake location and thus prevents or reduces the probability of liquid clogging the gas path while in use or during transit.

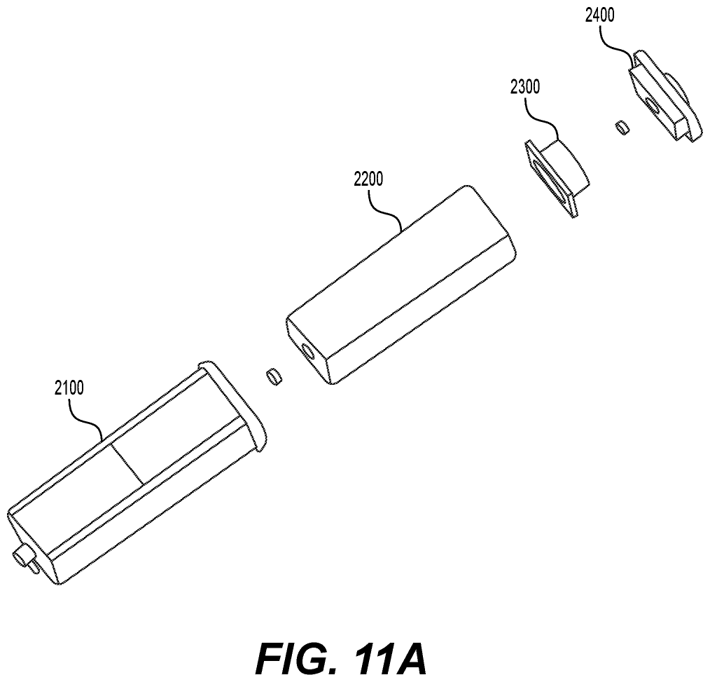

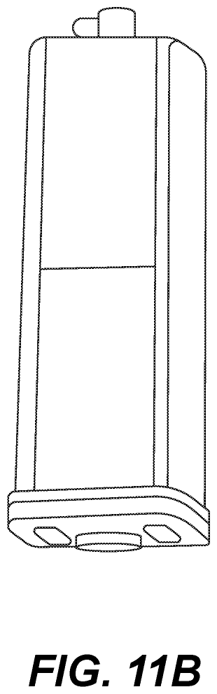

FIG. 11B is a perspective view of a cartridge assembly 2000, according to an aspect. FIG. 11A is an exploded view of the cartridge assembly 2000 of FIG. 11A. The cartridge assembly 2000 includes a cartridge housing 2100. The cartridge housing 2100 is filled with a porous media 2200 wetted with an antioxidant. A stopper 2300, which can be constructed of rubber or other suitable elastomeric material, can pack and contain the porous media 2200 and antioxidants within the cartridge housing 2100 under compression. Lid 2400 can be threadedly or otherwise coupled to the cartridge housing 2100. As shown best in FIGS. 4 and 5, the cartridge assembly 2100 can be disposed in the cassette 1000 discussed above.

The cartridge assembly 2000 can be fluidically coupled to the liquid vessel assembly 3000 via the capillary restrictor tubing 3211, tee fitting 3416, and cartridge inlet frit 2110. Input air gas port 3700 can allow air or other suitable carrier gas (e.g., oxygen, nitrogen, etc.) to flow from outside the cassette 1000 and to tee fitting 3416. By controlling the temperature of the reservoir of the dinitrogen tetroxide-containing liquid vessel assembly 3000, a rate at which nitrogen dioxide flows into tee fitting 3416 can be controlled. By controlling a flow rate of the carrier gas, a concentration of nitrogen dioxide delivered to the cartridge assembly 2000 via the inlet frit 2110 can be controlled.

The cartridge media 2200 can be silica gel or other suitable high-surface area wettable material. The cartridge media 2200 can be wetted with an antioxidant, such as an aqueous solution of ascorbic acid. In some aspects, the cartridge media 2200 can be wetted with water. Nitrogen dioxide can react with water bound on the cartridge media 2200 to produce nitric oxide following the following reactions:

a.6NO₂(gas)+3H₂O(liq.)→3HNO₃(liq.)+3HNO₂(liq.) Eq. 1a

b.3HNO₂(liq.)→HNO₃(liq.)+2NO(gas)+H2O(liq.) Eq. 1b

Ascorbic acid-wetted cartridge media 2200 (or other suitable antioxidant-containing cartridges) can be functionally similar to the media described in U.S. Pat. No. 8,607,785, the entire disclosure of which is hereby incorporated by reference. Cartridge assembly 2100 differs from known cartridges in that, in some aspects and excluding water and antioxidants, the cartridge media is substantially entirely (>95%) active derivatized silica gel. Similarly stated, the cartridge media may be substantially devoid (<5%) of non-active components, such as ultra-high molecular weight polyethylene binder material, which in some known cartridges was sintered with silica to produce solid media units. Such solid media units were not conformal to the known cartridge housings and, as such, did not maximize space within the housing. Cartridge media 2200, by contrast, is a flowable granular material. The stopper 2300 can keep the cartridge media 2200 in compression, causing the cartridge media 2200 to conform to the cartridge housing 2100, maximizing the use of available volume and obviating the need for a binder to produce a solid media unit. Furthermore, unlike known media units, which require multiple coating and drying steps on various individual components and sub-components, the cartridge media 2200 is more amenable to high-volume manufacturing, as the cartridge media 2200 can be wetted with an antioxidant solution or water in bulk. The cartridge media 2200 can then be added to cartridge housings in an assembly line-like process from a bulk container.

In some instances, cartridge 2100 may not include ascorbic acid or other antioxidants, which may degrade over time and instead rely on stable water to improve shelf life. In addition or alternatively, cartridge 2100 can be designed taking into account the availability of water to reduce nitrogen dioxide such that cartridge 2100 can be operable to produce nitric oxide based on a reaction with ascorbic acid (or other antioxidant) and/or water, which can improve the cartridge's capacity to produce nitric oxide and/or shelf life.

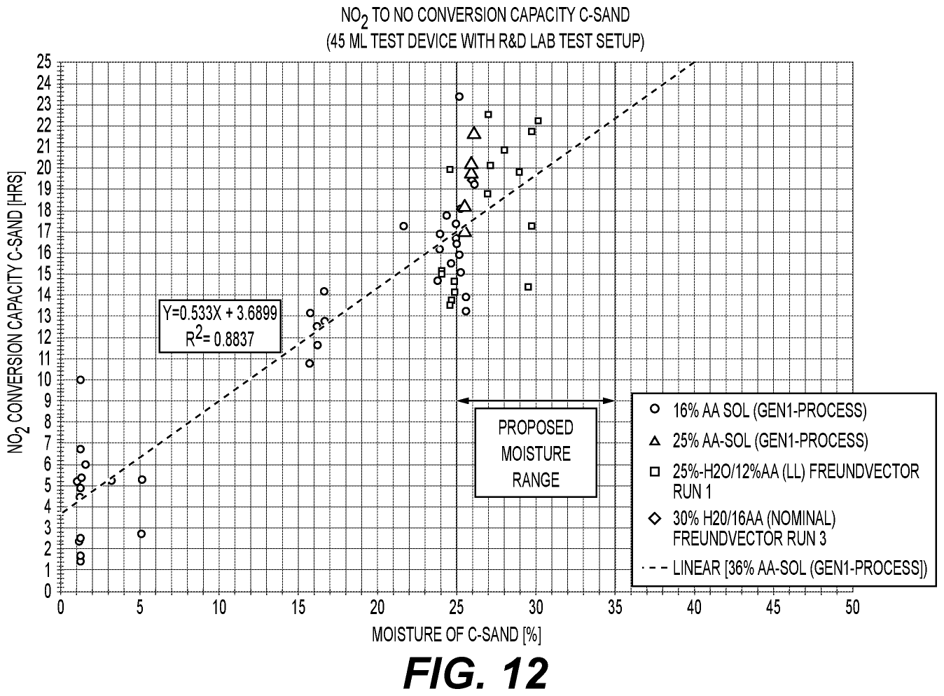

In addition, cartridge media 2200 may have higher density and/or higher moisture content than known cartridges. For example, some known cartridges have cartridge media with a water content of 1-10.6%. As shown in FIG. 12, experimental data reveals a positive correlation between cartridge media 2200 water content and nitrogen dioxide conversion capacity. The increased NO₂ conversion capacity is attributed to the reaction between NO₂ and water to produce NO (as discussed above), which results in increased NO₂ conversion capacity. In some aspects, cartridge media 2200 can have a water content of at least 20%. For example, the water content can be 20 wt % to 60 wt %, 20 wt % to 40 wt %, or 20 wt % to 35 wt %. Such high water concentrations, particularly those above 20 wt %, were infeasible with known cartridges, as the presence of inactive binder materials reduced the relative proportion of surface active material available to absorb water.

The cartridge media 2200 can be wetted with a solution containing suitable one or more antioxidants. It is desirable that the cartridge media 2200 and/or the material it is wetted with be non-toxic and/or food-safe, as the produced nitric oxide may be intended for inhalation. Accordingly, in such aspects, catalysts and/or antioxidants containing heavy metals or other materials that may contaminate the nitric oxide may be unsuitable. One suitable antioxidant is ascorbic acid. Various aspects of the cartridge media 2200 may be wetted with a solution containing between 0 and 32% ascorbic acid, including exemplary values of 1 wt %, 5 wt %, 10 wt %, 15 wt %, 20 wt %, 25 wt %, and 30 wt %. It is understood that ascorbic acid can be present in any amount that falls between any two disclosed above values, or it can be present in any range that can be formed by any two values that fall within the broadest range. For example, the ascorbic acid can be present in an amount of 1 wt % to 32 wt %, or 5 wt % to 32 wt %, or 15 wt % to 32 wt %, 1 wt % to 15 wt %, or 10 wt % to 15 wt %, and so on. It may be desirable for the antioxidant concentration to be at or below the room temperature solubility limit because, at higher concentrations, the antioxidant may precipitate out before the cartridge media 2200 is uniformly saturated, which could produce inconsistent nitric oxide conversion dynamics. It should be understood, however, that it may be possible to further increase the concentration of antioxidant by heating an antioxidant solution before wetting the cartridge media 2200.

### System

The currently known NO concentration (amount)-based control method has serious drawbacks. For example, the NO concentration-based control method may be unable to determine the optimal initial injection concentration at the start of dosing because the initial NO concentration is zero, and the flow supplied by a breathing-assisting device, such as a ventilator, is initially unknown. Known systems typically ask the user to provide the ventilator flow in ranges of Low, Medium, or High when the dose setpoint is entered prior to the start of dosing. The user-provided ventilator flow information is used to define normal initial injection parameters and rate of change for feedback control information within the algorithm, to maintain the dose without excessive overshoot of the target dose and to optimize the time to reach the target dose.

Still further, the NO concentration-based control method rate of feedback control may be constrained by the time response of the NO gas sensors to changes in NO concentration and the time for changes in the initial NO injection control to propagate from the source to the sample system. Depending on the breathing-assisting device, such as a ventilator, flow mode of use, the feedback control time interval can vary from less than 30 seconds to greater than 60 seconds. The rate of system parameter change is limited to the time of changes in information. For this reason, the system response to changes in the ventilator system operation is slower compared to a system that uses a faster-changing parameter to control the dose.

Other known nitric oxide delivery systems use the measured ventilator flow at the nitric oxide injection site as the primary input parameter to determine the injection flow from the delivery system with a fixed nitric oxide source concentration. This method is particularly well suited for the speed of system control with changes in ventilator system flow. Also, under normal operating conditions where all ventilator flow passes one time through the NO injector module, this control method achieves acceptable NO concentration accuracy (within +/−20% of the dose).

Such a system's accuracy can be insufficient in some important medical applications that require a very controlled presence of nitric oxide. The systems disclosed herein allow the achievement of this goal.

In some aspects, disclosed is a system for delivering a nitric oxide to a subject, wherein the system comprises a nitric oxide injection line configured to inject a first gas at an injection point into a breathing conduit comprising a breathing gas. In such exemplary and unlimiting aspects, the first gas comprises a first amount of nitric oxide. It is understood that the injection point can be anywhere along the breathing conduit. In some aspects, the injection point is abutting a portion of the breathing conduit proximal to a breathing-assisting device. It is understood that the breathing-assisting device can be any device that can provide a steady amount of breathing gas to the patient at the desired rate and concentration. In certain aspects, the breathing-assisting device is a ventilator. In still further aspects, the ventilator can be a face-mask ventilator. Yet, in other aspects, the ventilator can be a mechanical ventilator. In still further aspects, the mechanical ventilator can be a negative-pressure ventilator and/or a positive-pressure ventilator. Yet in other aspects, other types of ventilators can also be used for delivering the breathing gas to the patient. For example, other types of ventilators can include tracheostomy ventilators.

In yet still further aspects, the breathing gas can comprise air and/or oxygen-enriched air.

In still further aspects, the disclosed system comprises a sampling line configured to sample a second gas. In such aspects, the second gas comprises a second amount of nitric oxide and the breathing gas. It is understood that the sampling of the second gas can occur at any sampling location between the injection point of the first gas and a subject. In some aspects, the sampling location is closer to the subject than to the injection point.

In still further aspects, it is understood that the breathing-assisting device, in addition to the breathing conduit that is configured to deliver fresh breathing gas to the subject, can also comprise an exhaling conduit configured to remove the exhaled gas from the subject. In some aspects, the exhaled gas is vented into the ambient atmosphere, while in other aspects, the exhaled gas can be collected, scrubbed to remove carbon dioxide and other harmful gases and recirculated if needed. In a still further aspect, the breathing conduit and exhaling conduit can be attached to a face mask or a tube that is directly inserted into the subject. In certain aspects, the sampling location can be at a distance of about 6 to about 12 inches from where the breathing conduit and exhaling conduits connect to the face mask or a breathing tube. In yet other aspects, the distance can be from about 6 inches to about 12 inches, including exemplary values of about 7 inches, about 8 inches, about 9 inches, about 10 inches, and about 11 inches. It is understood that the distance can be anywhere between two of the disclosed above values. Yet, in still further aspects, the sampling location can be at a distance that can fall within any range formed by any of the above values. For example, and without limitations, the sample location can be at a distance between about 6 inches and 10 inches, or between 6 inches and 11 inches, or between 7 inches and 12 inches, or between 7 inches and 10 inches, or between 6.5 inches and 11.8 inches, and so on.

In yet other aspects, the system can further comprise a feedback-loop controller that is in communication with: (i) a nitric oxide set-point controller configured to set a nitric oxide set-point amount; (ii) a source configured to provide a third gas having a third amount of nitric oxide; (iii) at least one sensor configured to measure the second amount of nitric oxide in the sampling line, wherein the third amount of nitric oxide is determined by the feedback-loop controller based on the second amount of nitric oxide and the nitric oxide set-point amount.

In such exemplary and unlimiting aspects, the source configured to provide the third gas having the third amount of nitric oxide be any source configured to provide or form nitric oxide. In certain aspects, any of the nitric oxide sources disclosed herein can be utilized. In certain aspects, the nitric oxide source can be a tank of nitric oxide. In other aspects, the nitric oxide source can be a system that allows the formation of nitric oxide through a spark. Yet, in other aspects, the source of nitric oxide can be one or more cassettes configured to form nitric oxide through chemical reactions, as described above.

In still further aspects, the nitric-oxide set-point controller is configured to set a nitric oxide set-point amount or a target amount of nitric oxide that will be delivered to the subject. It is understood that the terms “target amount” and “nitric oxide set-point” can be used interchangeably hereby. The target (or set-point) amount is determined based on the subject's medical conditions and according to the doctor's prescription. In certain aspects, the target amount can be set manually by a caregiver or the subject. It is understood that the set-point can be defined in any term. For example, in some aspects, the nitric oxide set-point is a flow rate of nitric oxide that correlates to the first amount of nitric oxide. Yet, in other aspects, the nitric oxide set-point is a concentration of nitric oxide that correlates to the first amount of nitric oxide. It is understood that in certain aspects, the system can comprise set-point controllers that can allow defining the target amount both in terms of the nitric oxide flow rate and in terms of concentration of nitric oxide.

It is understood that the concentration (or amount) of nitric oxide can be determined in ppm units. In still further aspects, the systems disclosed herein are capable of delivering any desired amount of nitric oxide to the subject. In certain aspects, the amount of nitric oxide delivered to the subject can be greater than 0 ppm to about 10,000 ppm, including exemplary values of about 100 ppb, about 200 ppb, about 500 ppb, about 1 ppm, about 5 ppm, about 10 ppm, about 20 ppm, about 40 ppm, about 50 ppm, about 100 ppm, about 250 ppm, about 500 ppm, about 1,000 ppm, about 1,250 ppm, about 1,500 ppm, about 1,750 ppm, about 2,000 ppm, about 2,250 ppm, about 2,500 ppm, about 2,750 ppm, about 3,000 ppm, about 3,250 ppm, about 3,500 ppm, about 3,750 ppm, and about 4,000 ppm. It is understood that the actual amount of nitric oxide delivered to the subject can have any value falling between any two disclosed above values, or it can fall within any range formed by any values within the broadest range. In some examples, the nitric oxide delivered to the subject can be in a range of about 0.1 ppm to 100 ppm, e.g., for selective pulmonary vasodilation. In some examples, the nitric oxide delivered to the subject can be in a range of about 100 ppm to 300 ppm, e.g., for antimicrobial applications.

Yet, in other aspects, the target value can be determined in flow rate units and can be from about 0.5 microliters/min to about 2 milliliters/min of NO.

In still further aspects, the first gas injected into the breathing conduit can comprise a carrier gas. In such aspects, it is understood that the carrier gas is the gas that carries an amount (in this case, the first amount) of nitric oxide to the subject. In certain aspects, the carrier gas can go through the source of the nitric oxide and collect the nitric oxide with it regardless of the method of forming or delivering nitric oxide. Yet in other aspects, for example, if the source of nitric oxide is a tank comprising nitric oxide, such a tank can also comprise carrier gas. In certain aspects, the carrier gas can comprise an amount of inert gas such as nitrogen, or it can comprise air or oxygen-enriched air.

In still further aspects, the first gas, after injection into the breathing gas together with the breathing gas, can form the second gas. This second gas can comprise a second amount of nitric oxide, the breathing gas, and, optionally, carrier gas.

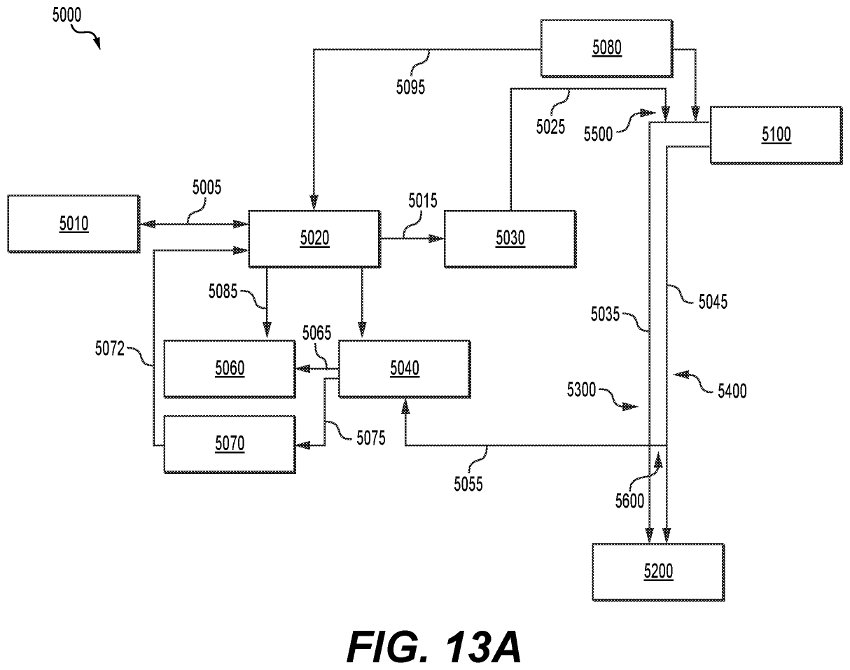

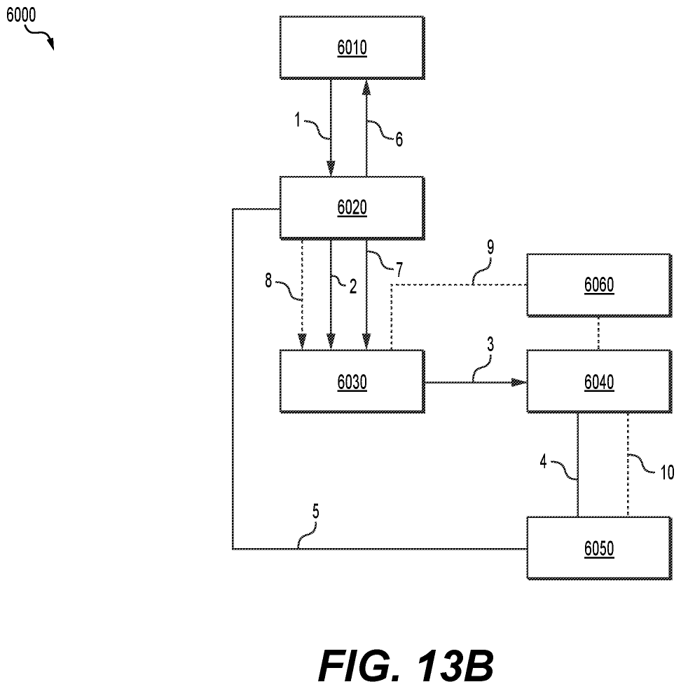

An exemplary system 5000 is shown in FIG. 13A. The breathing-assisting device 5100 has two conduits: a breathing conduit 5300 and an exhaling conduit 5400. The user can establish the target amount of the nitric oxide to be delivered in the nitric oxide set-point controller 5010, which communicates with the feedback-loop controller 5020, which then communicates with a source of nitric oxide 5030. The source of nitric oxide either generates the nitric oxide or delivers nitric oxide in a third amount. It is understood that at least once during nitric oxide delivery to the subject, the first amount of nitric oxide is the same as the third amount of nitric oxide. For example, at a first injection time, the controller 5020 communicates to the nitric oxide source 5030 to deliver an amount of nitric oxide to the breathing conduit 5600 at a delivery point of 5500, at this time, the third amount of nitric oxide equals the first amount of nitric oxide.

The breathing conduit 5300 carries the second gas 5035, which comprises the breathing gas and a second amount of nitric oxide. The exhaling conduit 5400 carries the gas that is exhaled by the subject. In some aspects, the exhaled gas is vented into the atmosphere. Yet in other aspects, the exhaled gas is collected and scrubbed of harmful gases such as carbon dioxide and recirculated back into the breathing conduit.

In still further aspects, the sampling line 5055 can be operated by a pump 5040. The sampling line samples the second amount of the nitric oxide at a location 5600, for example, of the breathing conduit 5300. It is understood that this schematic is only exemplary, and the sampling location can be anywhere, as disclosed above. The sampling line 5055, operated by pump 5040, carries the second gas by line 5065 to at least one sensor 5060. In such aspects, the at least one sensor is a sensor configured to determine the second amount of nitric oxide in the second gas. It is understood that any sensors capable of detecting nitric oxide can be utilized. The NO sensor 5060 is in communication with the feedback-loop controller 5020. The feedback-loop controller 5020, upon receiving the information about the second amount of nitric oxide in the sampling line, can communicate with the nitric-oxide set point controller 5010 to compare the second amount of nitric oxide with the target amount. In aspects where the second amount of nitric oxide is different from the nitric oxide set-point amount, the feedback-loop controller 5020 is configured to communicate with the source 5030 to adjust the third amount of nitric oxide to match the nitric oxide set-point amount.

In still further aspects and as disclosed above, the nitric oxide can form nitric oxide in-situ. It can be formed through a spark or chemical reaction using a liquid dinitrogen tetroxide. In aspects where the nitric oxide is used from the liquid dinitrogen tetroxide, the source can be any of the disclosed above. In still further aspects, and as disclosed above, the amount of nitric oxide formed by the source can be controlled by a temperature and pressure that is applied to the source.

An exemplary and unlimiting flow diagram 6000 of the disclosed herein system operation is shown in FIG. 13B. First, the user can establish a target amount of NO in 6010; this target amount is communicated 1 to the feedback-loop controller 6020. The feedback-loop controller communicates 2 with the NO source 6030, and a first amount 6040 of NO is injected 3 into the breathing conduit (not shown). The sampling line measures 4 a second amount of NO 6050 and communicates 5 it back to the feedback-loop controller 6020. If the target NO amount in 6010 is different from the second amount 6050, the controller 6020 communicates with the source 6030 to form 9 a third amount of NO 6060 to correct the overall amount of NO that reaches the subject. This amount is injected into the system and again measured at 10. The loop can continue this way as long as the subject receives NO.

In still further aspects, the system can further comprise an additional one or more sensors 5070. In such aspects, the additional one or more sensors are in communication 5072 with the feedback-loop controller. In still further aspects, the additional one or more sensors comprise an oxygen sensor and/or a nitrogen dioxide sensor. In yet other aspects, the oxygen sensor detects an amount of oxygen and/or the nitrogen dioxide sensor detects an amount of nitrogen dioxide in the second gas.

In still further aspects, the system can further comprise an auxiliary sensor 5080 positioned adjacent to an injection point and configured to measure a flow rate of the breathing gas, and wherein the auxiliary sensor is in communication 5095 with the feedback-loop controller. In such aspects, when the flow rate of the breathing gas is different than a flow rate correlated to the nitric oxide set-point amount, the feedback-loop controller corrects a flow of the third gas.

For example, in such aspects, the auxiliary sensor, such as a flow sensor 5080, is connected to an output of the breathing-assisting device, such as a ventilator. In such aspects, this sensor can provide a direct measurement of the ventilator dilution flow. If the auxiliary flow sensor is used, it provides the ventilator flow information to the system, which is used with the NO dose setpoint to define the target NO amount and the first (injection) amount of NO. Also, the flow sensor can be further used to detect changes in ventilator flow, which allows the system to respond to changes in ventilator flow more quickly than when using only the measured NO concentration in the circuit.

The systems that rely only on a flow sensor without sampling the amount of nitric oxide close to the subject can be insufficient when all ventilator flow does not pass one time through the NO injection. For example, such systems can be problematic if one needs to deliver NO to the subject during anesthesia. Anesthesia can be an expansive procedure, and therefore, it is desirable to recirculate the expiratory gas and, thus, to recirculate the anesthetic compounds back to the subjects. Anesthesia gas machines commonly recirculate an expiratory gas to minimize the utilization of costly anesthetic agents. When residual NO is present in the recirculated gas, the additional NO injection required to maintain the target concentration should be reduced compared to the normal use case without recirculation. The ventilator flow-based control method assumes the starting concentration of NO in the patient gas is zero and does not take the residual NO in the recirculated gas into account. As a result, the amount of NO to be injected can be overestimated as the actual amount of NO in the breathing conduit is higher than the target. This condition may continue to increase over time, leading to ventilator circuit NO and NO₂ concentrations that do not meet FDA Guidance requirements when recirculation occurs. As a result, NO delivery systems that use ventilator flow-based controls are not validated for use with circle anesthesia delivery systems.

Some ventilator systems (e.g., the Intrapulmonary Percussive Ventilation (IPV) system) introduce breathing gas downstream of the first gas injection. For this reason, the breathing gas flow measurement at the NO injection point does not represent the complete breathing gas flow to the patient. If the first amount of NO (that is being injected) is determined based on the flow measurement at the injection point, it can result in a lower NO concentration obtained by the subject than the desired set dose.

In the bi-directional flow modes of delivery, the accuracy of the ventilator circuit flow measurement is also reduced if flow in both directions is included in the control of the dose. This form of error with bi-directional flow devices results in an over-delivery of NO and ventilator circuit NO concentrations that do not meet FDA Guidance requirements.

The systems disclosed herein that are configured to measure the second amount of NO to be delivered to the subject and to correct it if needed by measuring the flow rate of the breathing gas can allow accurate delivery of NO amounts in anesthesia gas delivery systems, bidirectional flow systems (e.g., BiPAP, Phasitron), systems which introduce additional flow after the injection module (e.g., Intrapulmonary Percussion Ventilator) and so on.

In still further aspects, and as disclosed above, the system described herein can be used to deliver additional pharmaceutically active compounds, such as anesthetic materials. In such exemplary and unlimiting aspects, the breathing gas can comprise a first amount of a pharmaceutically active ingredient.

In still further aspects, the system can also measure the gas exhaled by the subjects. In some aspects, the exhaling conduit comprises a fourth gas exhaled by the subject, and wherein the fourth gas is vented to the ambient environment. Yet in still further aspects, the exhaling conduit can comprise a fourth gas exhaled by the subject, and wherein the system is configured to recirculate at least a portion of the fourth gas into the breathing conduit, wherein the at least a portion of the fourth gas is substantially free of carbon dioxide. It is understood that the fourth gas can be processed to remove carbon dioxide prior to recirculating it back into the breathing conduit.

In aspects where the fourth gas is recirculated, such gas can comprise a fourth amount of nitric oxide. The fourth gas enters the breathing conduit together with the breathing gas and the first gas to form the second gas that can be sampled at the sampling point. In such aspects, the second gas can comprise at least a portion of the fourth gas. It is understood that in such exemplary and unlimiting aspects, the second amount of nitric oxide comprises at least the first amount and the fourth amount of the nitric oxide.

If the breathing gas comprises a first amount of a pharmaceutically active ingredient and if the exhaling gas is collected, this exhaling gas (or fourth gas) can comprise a second amount of the pharmaceutically active ingredient. It is understood that in such aspects, the second amount of the pharmaceutically active ingredient is less than the first amount of the pharmaceutically active ingredient.

If the system is used for anesthesia, the pharmaceutically active ingredient comprises an anesthetic.

In still further aspects, the system disclosed herein is configured to deliver about 0.1 L/min to about 200 L/min of the breathing gas, including exemplary values of about 0.5 L/min, about 1 L/min, about 1.5 L/min, about 2 L/min, about 5 L/min, about 10 L/min, about 25 L/min, about 50 L/min, about 75 L/min, about 100 L/min, about 125 L/min, about 150 L/min, and about 175 L/min. It is further understood that the system can deliver any amount of the breathing gas that falls between any two foregoing values or between any ranges that any two foregoing values can form. For example, the system can deliver the breathing gas in an amount of about 0.1 L/min to about 195 L/min, or about 0.1 L/min to about 140 L/min, or about 0.5 L/min to about 100 L/min, or about 0.1 L/min to about 50 L/min, or about 0.1 L/min to about 100 L/min, or about 10 L/min to about 20 L/min, and so on.

Also disclosed herein is a setup comprising any of the disclosed systems integrated with a ventilator, an anesthesia gas delivery system, a bidirectional flow system, an intrapulmonary percussion ventilator system, a high-flow oxygen delivery system or any combination thereof.

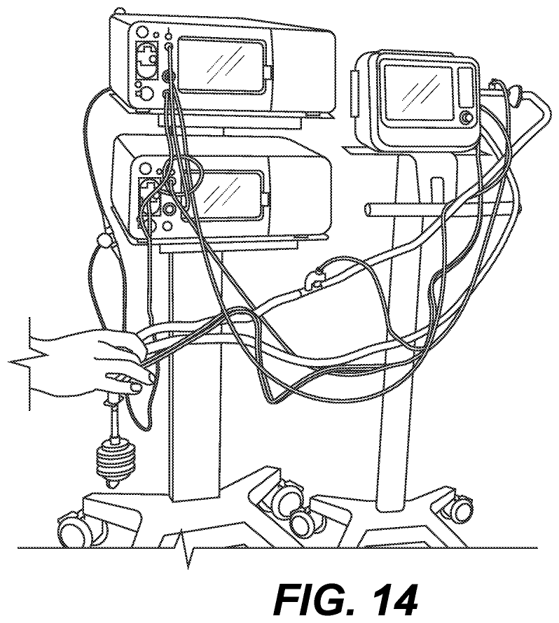

FIG. 14 shows an exemplary and unlimiting schematic of the setup disclosed herein in one aspect.

### Method

Still further discloses herein is a method of delivering nitric oxide to a subject. In such aspects, a method comprises: routing air from at least one air inlet through a first cassette that produces nitric oxide gas; detecting a fill state of the first cassette; and wherein the fill state of the first cassette is close to or below a threshold value, automatically re-routing air from the at least one air inlet through a second cassette that produces nitric oxide.

Any of the disclosed above cassettes can be used in this method. In certain aspects, the first cassette is disposed in a first receptacle, and the second cassette is disposed in a second separate receptacle. In yet other aspects, the first cassette and the second cassette are in electric communication with a controller. Again, it is understood that any of the disclosed above controllers can be used for the described method.

In still further aspects, the controller is configured to automatically re-route air. Yet in still further aspects, the controller is configured to connect both receptacles to an air inlet simultaneously if needed. In still further aspects, and as disclosed above, the controller used in the described method is configured to control a production rate of nitric oxide gas in the first cassette and/or second cassette.

While various aspects have been described above, it should be understood that they have been presented by way of example only and not limitation. Furthermore, although various aspects have been described as having particular features and/or combinations of components, other aspects possibly have a combination of any features and/or components from any of the aspects where appropriate, as well as additional features and/or components.

Where methods described above indicate certain events occurring in a certain order, the ordering of certain events may be modified. Additionally, certain of the events may be performed concurrently in a parallel process when possible, as well as performed sequentially as described above. Although various aspects have been described as having particular features and/or combinations of components, other aspects possibly have a combination of any features and/or components from any of the aspects where appropriate.

### EXEMPLARY ASPECTS

Example 1. A console comprising: at least one air inlet; a first receptacle configured to host a first source of nitric oxide; a second receptacle configured to host a second source of nitric oxide; a controller configured to selectively couple the at least one air inlet to one of the first receptacle or the second receptacle to deliver the nitric oxide from the first or the second source; and an outlet coupled to the first and/or second receptacle and configured to deliver nitric oxide to a subject.

Example 2. The console of any one of the examples herein, particularly Example 1, wherein the first source of nitric oxide comprises a first cassette configured to form nitric oxide and/or wherein the second source of nitric oxide comprises a second cassette configured to generate nitric oxide.

Example 3. The console of any one of the examples herein, particularly Examples 1 or 2, wherein when the at least one air inlet is coupled to the first receptacle and the first source of nitric oxide is substantially depleted, the controller is configured to automatically switch coupling of the at least one air inlet from the first receptacle to the second receptacle thereby delivering nitric oxide from the second source.

Example 4. The console of any one of the examples herein, particularly Examples 1-3, wherein at least once the at least one air inlet is coupled to the first receptacle and the second receptacle simultaneously.

Example 5. The console of any one of the examples herein, particularly Examples 1-4, wherein the controller is configured to monitor a fill state of the first and/or second nitric oxide source.

Example 6. The console of any one of the examples herein, particularly Examples 1-5, wherein the console comprises two or more air inlets, such that the first receptacle and the second receptacle are separately coupled to at least one air inlet.

Example 7. The console of any one of the examples herein, particularly Example 6, wherein the controller is configured to selectively activate the at least one air inlet in the first receptacle or the second receptacle based on a fill state of the first and/or second nitric oxide source.

Example 8. The console of any one of the examples herein, particularly Examples 1-7, wherein the controller is configured to control a rate of nitric oxide delivered to a subject.

Example 9. The console of any one of the examples herein, particularly Examples 2-8, wherein the controller is configured to control a rate of nitric oxide generation in the first cassette and/or second cassette.

Example 10. The console of any one of the examples herein, particularly Examples 2-9, wherein the first and/or second cassette comprises a first reservoir and a second reservoir, respectively, wherein the first and the second reservoir comprise dinitrogen tetroxide, and wherein the first and/or second cassette are configured to convert the dinitrogen tetroxide into nitrogen oxide.

Example 11. The console of any one of the examples herein, particularly Example 10, wherein the controller is configured to control a fill-state of the dinitrogen tetroxide.

Example 12. The console of any one of the examples herein, particularly Examples 3-11, wherein the controller is configured to gradually increase a concentration of nitric oxide from the second source while still delivering nitric oxide from the first source.

Example 13. The console of any one of the examples herein, particularly Examples 3-12, wherein a remaining nitric oxide in the first source of nitric oxide is vented after the at least one air inlet is switched to the second receptacle.

Example 14. A method comprising: routing air from at least one air inlet through a first cassette that produces nitric oxide gas; detecting a fill state of the first cassette; and wherein the fill state of the first cassette is close to or below a threshold value, automatically re-routing air from the at least one air inlet through a second cassette that produces nitric oxide.

Example 15. The method of any one of the examples herein, particularly Example 14, wherein the first cassette is disposed in a first receptacle and the second cassette is disposed in a second separate receptacle.

Example 16. The method of any one of the examples herein, particularly Example 14 or 15, wherein the first cassette and the second cassette are in electric communication with a controller.

Example 17. The method of any one of the examples herein, particularly Example 16, wherein the controller is configured to automatically re-route air.

Example 18. The method of any one of the examples herein, particularly Example 16 or 17, wherein the controller is configured to control a production rate of nitric oxide gas in the first cassette and/or second cassette.

Example 19. The method of any one of the examples herein, particularly Examples 14-18, wherein the re-routing is gradual or immediate.

Example 20. An apparatus, comprising: a frangible vessel containing dinitrogen tetroxide; a reservoir, the frangible vessel disposed within the reservoir, the reservoir configured to contain the dinitrogen tetroxide when the frangible vessel is broken; and an outlet, the outlet disposed above a wall of the reservoir, the frangible vessel, the reservoir, and the outlet collectively configured such that when the frangible vessel is broken, a level of the dinitrogen tetroxide in the reservoir does not reach the outlet regardless of an orientation of the vessel.

Example 21. The apparatus of any one of the examples herein, particularly Example 20, wherein the apparatus comprises a separator disposed in the outlet and positioned above dinitrogen tetroxide regardless of the orientation of the vessel or external vibration applied to the vessel.

Example 22. The apparatus of Example 19 or 20, wherein the reservoir is configured to be heated to form nitric dioxide from dinitrogen tetroxide.

Example 23. The apparatus of any one of the examples herein, particularly Example 22, wherein apparatus is further configured to convert nitric dioxide to nitric oxide.

Example 24. The apparatus of any one of the examples herein, particularly Examples 20-23, wherein the apparatus is thermally insulated.

Example 25. The apparatus of any one of the examples herein, particularly Examples 20-24, wherein the apparatus comprises an activation mechanism comprising an activation wedge configured to break the frangible vessel.

Example 26. The apparatus of any one of the examples herein, particularly Example 24 or 25, wherein the activation mechanism further comprises at least one activation sleeve, wherein the at least one activation sleeve is configured to maintain a positioning of the frangible vessel.

Example 27. A system for delivering a nitric oxide to a subject, wherein the system comprises: (a) a nitric oxide injection line configured to inject a first gas at an injection point into a breathing conduit comprising a breathing gas, wherein the first gas comprises a first amount of nitric oxide; (b) a sampling line configured to sample a second gas comprising a second amount of nitric oxide and the breathing gas at a sampling location between the injection point of the first gas and a subject and (c) a feedback-loop controller that is in communication with: (i) a nitric oxide set-point controller configured to set a nitric oxide set-point amount; (ii) a source configured to provide a third gas having a third amount of nitric oxide; (iii) at least one sensor configured to measure the second amount of nitric oxide in the sampling line; wherein the third amount of nitric oxide is determined by the feedback-loop controller based on the second amount of nitric oxide and the nitric oxide set-point amount.

Example 28. The system of any one of the examples herein, particularly Example 27, wherein the sampling location is at a distance of about 6 to about 12 inches from a connecting point of the breathing conduit and an exhaling conduit with a face mask or a breathing tube.

Example 29. The system of any one of the examples herein, particularly Example 27 or 28, wherein the sampling line is operated by a pump and is configured to deliver a predetermined amount of the second gas to the at least one sensor.

Example 30. The system of any one of the examples herein, particularly Examples 27-29, wherein the breathing gas is delivered by a breathing-assisting device.

Example 31. The system of any one of the examples herein, particularly Examples 27-30, wherein the breathing gas comprises air and/or oxygen-enriched air.

Example 32. The system of any one of the examples herein, particularly Examples 27-31, wherein the first gas further comprises a carrier gas.

Example 33. The system of any one of the examples herein, particularly Example 32, wherein the second gas further comprises the carrier gas.

Example 34. The system of any one of the examples herein, particularly Examples 27-33, wherein the at least one sensor is a NO-sensor.

Example 35. The system of any one of the examples herein, particularly Examples 27-34, wherein the feedback-loop controller is in further communication with additional one or more sensors.

Example 36. The system of any one of the examples herein, particularly Example 35, wherein the additional one or more sensors comprise an oxygen sensor and/or a nitrogen dioxide sensor.

Example 37. The system of any one of the examples herein, particularly Example 36, wherein the oxygen sensor detects an amount of oxygen and/or the nitrogen dioxide sensor detects an amount of nitrogen dioxide in the second gas.

Example 38. The system of any one of the examples herein, particularly Examples 27-37, wherein the nitric oxide set-point amount is a therapeutical target amount.

Example 39. The system of any one of the examples herein, particularly Examples 27-37, wherein the nitric oxide set-point is a flow rate of nitric oxide that correlates to the first amount of nitric oxide.

Example 40. The system of any one of the examples herein, particularly Examples 27-39, wherein the nitric oxide set-point is a concentration of nitric oxide that correlates to the first amount of nitric oxide.

Example 41. The system of any one of the examples herein, particularly Examples 27-40, wherein when the second amount of nitric oxide is different from the nitric oxide set-point amount, the feedback-loop controller is configured to communicate with the source to adjust the third amount of nitric oxide to match the nitric oxide set-point amount.

Example 42. The system of any one of the examples herein, particularly Examples 27-41, wherein at least once during nitric oxide delivery to the subject, the first amount of nitric oxide is the same as the third amount of nitric oxide.

Example 43. The system of any one of the examples herein, particularly Examples 27-42, wherein the source is configured to form nitric oxide in-situ.

Example 44. The system of any one of the examples herein, particularly Example 43, wherein the third amount is controlled by a temperature and pressure supplied to the source.

Example 45. The system of any one of the examples herein, particularly Example 27-44, wherein the system comprises an auxiliary sensor positioned adjacent to an injection point and configured to measure a flow rate of the breathing gas, and wherein the auxiliary sensor is in communication with the feedback-loop controller.

Example 46. The system of any one of the examples herein, particularly Example 45, wherein when the flow rate of the breathing gas is different than a flow rate correlated to the nitric oxide set-point amount, the feedback-loop controller corrects a flow of the third gas.

Example 47. The system of any one of the examples herein, particularly Examples 27-46, wherein the breathing gas further comprises a first amount of a pharmaceutically active ingredient.

Example 48. The system of any one of the examples herein, particularly Examples 27-47, wherein an exhaling conduit comprises a fourth gas exhaled by the subject and wherein the fourth gas is vented to the ambient environment.

Example 49. The system of any one of the examples herein, particularly Examples 27-48, wherein an exhaling conduit comprises a fourth gas exhaled by the subject and wherein the system is configured to recirculate at least a portion of the fourth gas into the breathing conduit, wherein the at least a portion of the fourth gas is substantially free of carbon dioxide.

Example 50. The system of any one of the examples herein, particularly Example 49, wherein the fourth gas comprises a fourth amount of nitric oxide.

Example 51. The system of any one of the examples herein, particularly Example 49 or 50, wherein the second gas comprises at least a portion of the fourth gas.

Example 52. The system of any one of the examples herein, particularly Example 51, wherein the second amount of nitric oxide comprises at least the first amount and the fourth amount of the nitric oxide.

Example 53. The system of any one of the examples herein, particularly Examples 49-52, wherein the fourth gas comprises a second amount of the pharmaceutically active ingredient, wherein the second amount of the pharmaceutically active ingredient is less than the first amount of the pharmaceutically active ingredient.

Example 54. The system of any one of the examples herein, particularly Examples 49-53, wherein the pharmaceutically active ingredient comprises an anesthetic.

Example 55. The system of any one of the examples herein, particularly Examples 27-24, wherein the system is configured to deliver 0.1 L/min-200 L/min of breathing gas.

Example 56. A setup comprising the system of any one of the examples herein, particularly Examples 27-55 integrated with a ventilator, an anesthesia gas delivery system, a bidirectional flow system, an intrapulmonary percussion ventilator system, a high-flow oxygen delivery system or any combination thereof.

What is claimed is:

1. A console comprising:
at least one air inlet;
a first receptacle configured to host a first source of nitric oxide;
a second receptacle configured to host a second source of nitric oxide;
a controller configured to selectively couple the at least one air inlet to one of the first receptacle or the second receptacle to deliver the nitric oxide from the first or the second source; and
an outlet coupled to the first and/or second receptacle and configured to deliver nitric oxide to a subject.

2. The console of claim 1, wherein the first source of nitric oxide comprises a first cassette configured to form nitric oxide and/or wherein the second source of nitric oxide comprises a second cassette configured to generate nitric oxide.

3. The console of claim 1, wherein when the at least one air inlet is coupled to the first receptacle and the first source of nitric oxide is substantially depleted, the controller is configured to automatically switch the coupling of at least one air inlet from the first receptacle to the second receptacle thereby delivering nitric oxide from the second source.

4. The console of claim 1, wherein the at least one air inlet is coupled to the first receptacle and the second receptacle simultaneously.

5. The console of claim 1, wherein the controller is configured to monitor a fill state of the first and/or second nitric oxide source.

6. The console of claim 1, wherein the console comprises two or more air inlets, such that the first receptacle and the second receptacle are separately coupled to at least one air inlet.

7. The console of claim 6, wherein the controller is configured to selectively activate the at least one air inlet in the first receptacle or the second receptacle based on a fill state of the first and/or second nitric oxide source.

8. The console of claim 1, wherein the controller is configured to control a rate of nitric oxide delivered to a subject.

9. The console of claim 2, wherein the controller is configured to control a rate of nitric oxide generation in the first cassette and/or second cassette.

10. The console of claim 2, wherein the first and/or second cassette comprise a first reservoir and a second reservoir, respectively, wherein the first and the second reservoir comprise dinitrogen tetroxide, and wherein the first and/or second cassette are configured to convert the dinitrogen tetroxide into nitrogen oxide.

11. The console of claim 10, wherein the controller is configured to control a fill-state of the dinitrogen tetroxide.

12. The console of claim 3, wherein the controller is configured to gradually increase a concentration of nitric oxide from the second source while still delivering nitric oxide from the first source.

13. The console of claim 3, wherein a remaining nitric oxide in the first source of nitric oxide is vented after the at least one air inlet is switched to the second receptacle.

14. A method comprising:
routing air from at least one air inlet through a first cassette that produces nitric oxide gas;
detecting a fill state of the first cassette; and
wherein the fill state of the first cassette is close to or below a threshold value, automatically re-routing air from the at least one air inlet through a second cassette that produces nitric oxide.

15. The method of claim 14, wherein the first cassette is disposed in a first receptacle and the second cassette is disposed in a second separate receptacle.

16. The method of claim 14, wherein the first cassette and the second cassette are in electric communication with a controller.

17. The method of claim 16, wherein the controller is configured to automatically re-route air.

18. The method of claim 16, wherein the controller is configured to control a production rate of nitric oxide gas in the first cassette and/or second cassette.

19. An apparatus comprising:
a frangible vessel containing dinitrogen tetroxide;
a reservoir, the frangible vessel disposed within the reservoir, the reservoir configured to contain the dinitrogen tetroxide when the frangible vessel is broken; and
an outlet, the outlet disposed above a wall of the reservoir,
the frangible vessel, the reservoir, and the outlet are collectively configured such that when the frangible vessel is broken, a level of the dinitrogen tetroxide in the reservoir does not reach the outlet, regardless of an orientation of the vessel.

20. The apparatus of claim 19, wherein the apparatus comprises a separator disposed in the outlet and positioned above dinitrogen tetroxide regardless of the orientation of the vessel or external vibration applied to the vessel.

21. The apparatus of claim 19, wherein the apparatus is thermally insulated.

22. The apparatus of claim 19, wherein the apparatus comprises an activation mechanism comprising an activation wedge configured to break the frangible vessel.

23. The apparatus of claim 22, wherein the activation mechanism further comprises at least one activation sleeve, wherein the at least one activation sleeve is configured to maintain a positioning of the frangible vessel.
